(19) United States
(12) Patent Application Publication
(10) Pub. No.: US 2024/0016939 A1
(43) Pub. Date: Jan. 18, 2024

(54) Liquid Composition Comprising Cereal Beta-Glucan or a Cereal Extract Comprising Beta-Glucan

(71) Applicant: Symrise AG, Holzminden (DE)

(72) Inventors: Mickaël Larnicol, Paris (FR); Léa Schmidt, Asnières-sur-Seine (FR); Marielle Le Maire, Boulogne Billancourt (FR)

(21) Appl. No.: 18/252,969

(22) PCT Filed: Nov. 17, 2020
(86) PCT No.: PCT/EP2020/082383

§ 371 Date: May 15, 2023

(51) Int. Cl. A61K 47/36; A61K 9/06; A61K 8/04; A61K 8/73; A61Q 19/00; A61K 45/06

(52) U.S. Cl. CPC A61K 47/36; A61K 9/06; A61K 8/042; A61K 8/73; A61Q 19/00; A61K 45/06

(57) ABSTRACT

The present invention relates generally to a liquid composition comprising cereal beta-glucan or a cereal extract comprising beta-glucan and an added inorganic and/or organic salt. Particularly, the present invention relates to a method for producing a liquid cereal beta-glucan comprising composition with retarded gelation and a method for transforming said gelled cereal beta-glucan comprising composition. Additionally, the present invention relates to said beta-glucan comprising composition as a food, food supplement, cosmetic, pharmaceutical or veterinary preparation. More particularly, the present invention pertains to a food, food supplement, cosmetic, pharmaceutical or veterinary preparation comprising said β-cereal comprising composition. Finally, the present invention relates to the use of certain inorganic and/or organic salts for retarding gelation of a cereal beta-glucan comprising composition.

### TECHNICAL FIELD

The present invention relates generally to a liquid composition comprising cereal beta-glucan or a cereal extract comprising beta-glucan and an added inorganic and/or organic salt. Particularly, the present invention relates to a method for producing a liquid cereal beta-glucan comprising composition with retarded gelation and a method for transforming said gelled cereal beta-glucan comprising composition. Additionally, the present invention relates to said beta-glucan comprising composition as a food, food supplement, cosmetic, pharmaceutical or veterinary preparation. More particularly, the present invention pertains to food, food supplement, cosmetic, pharmaceutical or veterinary preparations comprising said β-cereal comprising composition. Finally, the present invention relates to the use of certain inorganic and/or organic salts for retarding gelation of a cereal beta-glucan comprising composition.

### BACKGROUND ART

Poaceae, also known as Gramineae, is a large and nearly ubiquitous family of monocotyledonous flowering plants known as grasses. Grasses are an economically important family of plants. They have been grown as feed for domesticated animals for up to 6,000 years, and the grains of grasses such as wheat, rice, maize (corn), barley, sorghum, millet and oat have been and still are the most important human food crops.

Cereal grains are an excellent source of numerous unique substances among biologically active compounds such as dietary fibre (arabinoxylans, β-glucans, cellulose, lignin and lignans), sterols, tocopherols, tocotrienols, phenolic compounds, vitamins and microelements.

Beta-glucans are among the principal fractions of cereal grain dietary fibre. They occur in the walls of aleurone layer cells and bran. Average total β-glucan content in grains of barley, oat, rye and wheat is 4.0-7.0%, 2.2-7.8%, 1.2-2.9% and 0.4-1.4%, respectively.

Oats exist in two main species, Avena sativa L. and Avena nuda L. (synonyms include Avena sativa subsp. nuda (L.) after Gillet & Magne, and Avena sativa var. nuda (L.) after Koern). A. sativa, also known as common or husked oat, is primarily grown in cool temperate climates, especially in the cool and moist regions of Northern Europe and North America. A. nuda is known as naked or huskless oat because the husk is removed when the crop is harvested, and it has a free threshing character similar to wheat. Husked oats represent the majority of global oat production, except in China, where naked oat is the most common type.

In oats, a principal component of the soluble fibres comprises oat β-glucans. It is located in the endosperm cell walls, which are thickest adjacent to the aleurone layer, in the sub-aleurone layer. The process of isolating and purifying oat β-glucan is known in the art. The extraction methodologies for oat β-glucans are based on solubility in hot water and alkaline solutions. Purification can be achieved for example by separating the co-extracted proteins by isoelectric precipitation, and precipitating the β-glucan using ammonium sulphate, 2-propanol or ethanol. Other methodologies for oat beta-glucans are based on extraction with an aqueous alcohol or acetone solution and subsequent chromatographic enrichment of the beta-glucan.

Beta-glucan is classified as a viscous gum. Gums are either hydrophobic or hydrophilic high molecular weight substances that in an appropriate solvent produce gels or highly viscous suspensions or solutions at low dry substance content. Gums commonly used in food, medicine, and industrial products include starches, cellulose derivatives, guar gum, locust bean gum, pectin, algin, carrageenan, xanthan, beta-glucan, and gum arabic.

Most gums in a solid state consist of polysaccharide chains grouped in a disorganized manner. The random nature of this structure only partially satisfies the intermolecular interaction potential, for example hydrogen bonding potential is not saturated. The numerous unsatisfied hydrogen bonds are capable of rapid hydration, binding water molecules at hydrogen bonding positions not otherwise involved in intra-and intermolecular bonding of the polysaccharide molecules.

When a polysaccharide is placed in water, the water molecules quickly penetrate amorphous regions and bind to available polymer sites, competing for and eventually reducing other inter-polysaccharide bonds to negligible numbers.

Segments of a polysaccharide chain become fully solvated and move away by kinetic action, tearing apart more inter-polysaccharide bonds, which are immediately solvated. This intermediate stage in the dissolution of a polymer molecule represents a transient gel state and portrays a universal stage in the dissolution of all polysaccharides. For carbohydrates not fully soluble in cold water, dissolution is completed by heating with rapid mixing and results in a monodispersed hydrogel solution. Unless mechanisms are adopted to prevent reannealing, the hydrogel will form a gel on cooling.

When linear gum molecules in solution collide, they form an association over several chain units. At the junctions of the chains, molecular bonding e. g. hydrogen bonds form and as the interactions spread throughout the solution a great three-dimensional network is formed and the liquid becomes a gel. Once formed, a gel may undergo lengthening of junction zones as the molecules slide over each other or by moving together. This causes an overall tightening of network structure and a decrease in solvent filled spaces between molecules.

Hence, the solvent is exuded from the gel to produce syneresis or weeping. Cooling or freezing a colloidal solution or gel accelerates this effect.

Glucans are structural polysaccharides present in the cell wall of yeasts, bacteria, fungi, and cereals consisting only of glucose. However, since it is possible to link the glucose molecules in different stereochemical configurations, glucans are a diverse group of compounds with differing chemical, physical and functional properties.

The (1→3), (1→4) beta-D-glucans, which are commonly referred to as β-glucans, consist of D-glucopyranose residues linked by (1→4) beta glycosidic bonds which separate every two, three or four D-glucopyranose units by one (1→3) beta glycosidic linkage. Among the cereals, there are significant differences in the organisations of the (1→3) and (1→4) beta glycosidic linkages of the chain. The structure of beta-glucans dictates their physicochemical (solubility in water, viscosity and gel formation) and functional characteristics: the barley and oat glucans are quite soluble, but the wheat glucan is less so.

More particularly, the solubility properties of beta-glucans differ according to their source. For example, cereal beta-glucans are normally soluble in aqueous solvents, whereas yeast (Saccharomyces cerevisiae) beta-glucans are insoluble in aqueous solvents. Soluble glucans are desirable. Beta-glucans from oat having a high molecular weight (500 to 650 kDa) are the best water-soluble beta-glucan family.

Beta-glucan compounds have potent beneficial biological activities making them valuable and highly interesting natural active nutritional agents. The cosmetics industry favours the use of beta-glucan for its viscosity, shear strength and moisture enhancing properties. Additionally, beta-glucans have numerous benefits, such as reducing the occurrence of coronary heart disease, decreasing the level of LDL cholesterol and lipids in blood serum, reducing blood pressure, improving sensitivity to insulin and enabling the control of blood glucose levels, as well as antioxidant and anti-inflammatory activity.

Industrial gums are sold as powders because of problems with solution stability.

Low temperature leads to insufficient dissolution: the molecules are closely packed in the solution and unable to form a large continuous gel network that can entrap water. The optimal dissolution temperature differs significantly between the cereal beta-glucans: at beta-glucan gel at 37° C. while barley beta-glucan at 57° C.

In the course of time, especially during storage, the beta-glucan solutions get unstable and gel, at room temperature; the gel forms a block and water is released. In particular, unmodified oat (1→3), (1→4) beta-D-glucan, form highly viscous solutions in water at concentration >0.75%. At concentrations >1.2%, the solutions have the consistency of a thick hydrogel. Gelation is the process by which monomeric particles, such as particles present in a hydrosol, i.e. a dispersed or solubilized viscous aqueous preparation) combine with the continuous phase to form a polymeric hydrogel. Thus, gelation is the process of forming a colloid in which the dispersed phase (beta-glucan) is combined with the continuous phase (water or aqueous solvent) to produce a viscous gel-like product.

In practical use, however, stable liquid beta-glucan compositions are needed. More particularly, a beta-glucan composition is desired, in which the aggregation process of the beta-glucan molecules is delayed or gelation is even prevented.

Studies show that the dissolution of beta-glucans by heating the beta-glucan solution to 85° C. did not result in gelation. Heat breaks the gel. However, the state of beta-glucans in solution and their thermal treatment history affect their technological and physiological functionality. The rheological properties of beta-glucans may also be altered during the process and the storage.

Alternatively, for a gelled product, heating (40° C., 30 to 60 min) leads to the initial state, i.e. an aqueous gel product, without agglomeration or big bloc of gel. However, this is not acceptable for customers and hard to handle in production.

In order to produce or enhance solubility, and stabilize gel solutions, gums may be chemically modified. For example, addition of methyl, ethyl, carboxymethyl, hydroxyethyl, hydroxypropyl, phosphate, sulphate and similar groups enhances solubility and produces stable solutions of high viscosity. However, the chemical modification may change the molecular structure of the glucan and may also affect its efficacy.

U.S. Pat. No. 6,284,886 provides for a simple and efficient method of formulating and producing stable solutions of beta-glucan utilizing a biological, zwitterionic buffer during the purification process, to retard gelation and/or precipitation of the beta-glucan upon cooling.

Hence, there is a need in providing another stable, homogenous and easily storable liquid solution of beta-glucan where viscosity and appearance are stable over a long storage period and do not gel and, thus, shelf-life is improved. Furthermore, there is a need in a method of producing a stable, homogenous liquid solution of beta-glucan where gelation of the beta glucan upon cooling and/or during storage is retarded.

It was surprisingly discovered that a beta-glucan solution, where viscosity and appearance are stable over a long storage period, can be produced and aggregation process or gelation upon cooling and storage can be retarded by adding a certain inorganic and/or organic salt to the beta-glucan solution, as shown herein, provides for increased stability and retards the formation of gel.

The methods of the present invention employ the addition of a certain inorganic and/or organic salt, which provides for increased stability and extended shelf-life by retarding the formation of gels upon cooling and during storage. Additionally, without gelation the composition provides a clear solution which is an advantageous factor for use in consumer products.

### SUMMARY OF THE INVENTION

Accordingly, the primary aim of the present invention is therefore to provide a new liquid composition comprising or consisting of:
at least one cereal beta-glucan or a cereal extract comprising at least one cereal beta-glucan; and
at least one added inorganic and/or organic salt or a mixture thereof.

In a further aspect, the present invention relates to a method for producing a liquid cereal beta-glucan composition having retarded gelation, said method comprises
(a) providing a solution comprising or consisting of at least one cereal beta-glucan or a cereal extract comprising at least one cereal beta-glucan;
(b) adding to the solution of step (a) at least one inorganic and/or organic salt or a mixture thereof to obtain a mixture;
(c) optionally adjusting the pH value of the mixture to a pH value in a range from 3.0 to 7.0, in particular in a range from 4.0 to 6.0; and
(d) optionally heating said mixture to a temperature in a range from 20° C. to 100° C., in particular in a range from 50° C. to 80° C.

In a still further aspect, the present invention relates to a method for transforming a gelled cereal beta-glucan comprising composition, wherein said method comprises:
(i) providing a gelled composition comprising or consisting of a at least one cereal beta-glucan or a cereal extract comprising at least one cereal beta-glucan;
(ii) heating said gelled composition to a temperature in a range from 20° C. to 100° C., in particular in a range from 50° C. to 80° C., to obtain a solution;
(iii) adding to the solution of step (ii) at least one inorganic and/or organic salt or mixtures thereof to obtain a mixture; and
(iv) optionally adjusting the pH value of the mixture to a pH value in a range from 3.0 to 7.0, preferably in a range from 4.0 to 6.0.

In a further aspect, the present invention aims to provide a composition comprising a cereal beta-glucan or a cereal extract, obtainable by using the method according to the present invention.

A further aim of the present invention is the use of the composition as a cosmetic for skin care, scalp care, hair care, nail care or in the prevention and/or the treatment of skin conditions, intolerant and sensitive skin, skin irritation, skin reddening, wheals, pruritus (itching), skin aging, wrinkle formation, loss of skin volume, loss of skin elasticity, pigment spots, pigment abnormalities, or dry skin, i.e. for moisturising the skin.

In a still further aspect, the present invention relates to the composition according to the present invention for use as a medicament, in particular for use in the prevention and/or treatment of dermatological or keratological diseases, in particular of dermatological or keratological diseases having a barrier related, inflammatory, immunoallergic, atherogenic, xerotic or hyperproliferative component or in the prevention and/or treatment of dermatological diseases associated with increased ROS production or in the prevention and/or treatment of cardiovascular diseases, allergic reactions, coronary heart disease, for decreasing the level of LDL cholesterol and lipids in blood serum, for reducing blood pressure, for improving sensitivity to insulin and for enabling the control of blood glucose levels.

In yet another aspect, the present invention relates to food, food supplements, cosmetic, pharmaceutical or veterinary preparations comprising the beta-glucan composition according to the present invention.

Finally, the present invention is directed to the use of certain inorganic and/or organic salts for improving the stability of a cereal beta-glucan comprising composition and for retarding the gelation of a cereal beta-glucan comprising composition.

The invention is specified in the appended claims. The invention itself, and its preferred variants, other objects and advantages, are however also apparent from the following detailed description in conjunction with the accompanying examples and figures.

### DESCRIPTION OF FIGURES

FIG. 1 shows the appearance of three beta-glucan compositions with different pH values after 2 months storage at room temperature.

### DETAILED DESCRIPTION OF THE INVENTION

In a first aspect, the present invention relates to a new liquid composition comprising or consisting of:
at least one cereal beta-glucan or a cereal extract comprising at least one cereal beta-glucan; and
at least one added inorganic and/or organic salt or a mixture thereof.

The composition according to the present invention comprises at least one cereal beta-glucan or a cereal extract comprising at least one cereal beta-glucan as its main ingredient.

As used in this specification and the appended claims, the phrase “at least one of” means that the composition can comprise either one of the respective component or can comprise more of the respective component, i.e. two, three or even more of said respective component. Additionally, the phrase “at least one of”, when applied to a list, means any combination of the items specified in the list.

The term “cereal” means any several grain such as, but not limited to, cultivars of barley, oat, wheat, rye, sorghum, millet, and corn. In a preferred variant of the present invention, the cereal is oat.

The term “beta-glucan” means a glucan with a beta (1→3) linked glucopyranosyl backbone, or a beta (1→4) linked glucopyranosyl backbone, or a mixed beta ((1→3) (1→4) linked glucopyranosyl backbone.

A “cereal beta glucan” or a “cereal extract comprising at least one cereal beta-glucan” in the context of the present invention means a beta-glucan or a beta glucan extract, respectively, which is derived from a cereal source.

The composition according to the present invention comprises at least one beta-glucan compound. Beta-glucans are a group of high molecular 8-D-glucose polysaccharides which are commonly designated as beta-glucans and which naturally occur in the cell walls of cereals, bacteria and fungi and exhibit significantly different physiochemical properties depending on the source. In cereals (oat and barley) beta-glucans are composed of mixed-linkage (1->3) (1->4) beta-D-glucose units, while it is composed of mixed-linkage of (1->3) (1->6) beta D-glucose units in mushrooms and yeasts. The at least one beta-glucan compound is preferably, but not necessarily, a cereal beta-glucan.

Beta-glucans consist of D-glucopyranose residues linked by beta-(1->4) glycosidic bonds/linkages which separate every two, three or four D-glucopyranose unit by one beta-(1->3) glycosidic bond/linkage; cellulose like fragments consisting of three and four glucose residues are designated as DP3 and DP4, respectively.

The principal component of oat soluble fiber is the linear polysaccharide (1->3), (1->4) beta-D-glucan, usually called beta-glucan; these glucans have a molecular weight of 35 to 3100 kDA, a DP3 value of 54.2 to 60.9, a DP4 value of 33.8 to 36.7 and a DP3/DP4 ratio of 1.5 to 2.3.

Within the context of the present invention, the at least one beta-glucan is selected from beta-glucans selected from the group consisting of a glucan with a beta (1→3) linked glucopyranosyl backbone, or a glucan with a beta (1→4) linked glucopyranoyl backbone, or a glucan with a mixed beta (1→3) (1→4) linked glucopyranosyl backbone. In a preferred variant, the at least one beta-glucan is preferably a beta-glucan with a mixed β-(1->3)-β-(1->4)-linked glucopyranosyl backbone and having a different molecular weight, which are preferably derived from a cereal source.

The designation of beta (1→3) for a glycosidic linkage indicates that the etheric oxygen bridge between two consecutive monosaccharaide units of the polysaccharide connects the number 1 carbon of the first unit to the number 3 carbon of the second unit, and that etheric oxygen bridge attaches to carbon 1 of the first unit.

The designation of beta (1→4) for a glycosidic linkage indicates that the etheric oxygen bridge between two consecutive monosaccharaide units of the polysaccharide connects the number 1 carbon of the first unit to the number 4 carbon of the second unit, and that etheric oxygen bridge attaches to carbon 1 of the first unit.

Beta-glucan from any of several known cereal sources can be used in the composition according to the present invention. Such cereals include any of the cultivars of e.g. barley, oat, wheat, rye, corn, sorghum and millet. In a preferred variant of the present invention, the cereal is oat or barley, due to their high beta-glucan content. Still more preferred, the beta-glucan is derived from an oat source.

The beta-glucan of the composition of the present invention is either used in powdered form from commercial suppliers, such as Nutraland, Lantmännen, preferably as pure beta-glucan. Said powdered beta-glucan has a purity of more than 98%, and a low salt content.

The beta-glucan of the composition according to the present invention is preferably unmodified.

In an alternative variant, the beta-glucan is employed in the composition according to the present invention as an extract obtained from any of known cereal sources as specified above, comprising at least one beta-glucan.

Within the context of the present invention, the term “oat extract” is generally meant to encompass a compound or mixture of compounds obtained from oats. The extract can be obtained by extraction from any oat species, fresh or dried, or parts thereof, such as grains, husks, trichomes or oat straw. Altering the composition of the extracting solvent can change the extract composition, thereby enhancing or reducing its biological activity.

The naturally occurring beta-glucan or mixture of beta-glucan as described above, are obtained and isolated from the plant of the genus Avena by extraction, in particular from any oat species, fresh or dried, or parts thereof, such as milled grains, non-milled grains, husks, trichomes or oat straw of the oat species Avena sativa or Avena nuda.

The extract according to the first aspect of the present invention is preferably prepared from oats. The two main species of oats are Avena sativa L. and Avena nuda L. (synonyms include Avena sativa subsp. nuda (L.) after Gillet & Magne, and Avena sativa var. nuda (L.) after Körn). A. sativa is also known as common or husked oat. A. nuda is known as naked or huskless oat because the husk is removed when the crop is harvested. Oats can be processed and separated into constituent fractions including oat grains, husks and trichomes. In a preferred variant, the starting material for the oat extract is milled or non-milled grains of the species Avena sativa or Avena nuda or oat straw.

The extracting solvent (extractant) for favourably extracting the beta-glucan is selected from the group consisting of mixtures of water and an organic solvent, wherein the organic solvent is preferably a solvent suitable for foodstuffs or cosmetic or pharmaceutical preparations. It goes without saying that such solvents need be suitable for and compatible with the preparation of foods, cosmetics or pharmaceutical preparations.

In a more preferred variant, the extracting solvent comprises a mixture of water and an alcohol or acetone. The alcohol is preferably selected from the group consisting of methanol, ethanol, n-propanol, isopropanol, n-butanol, isobutanol, t-butanol and mixtures, i.e. combinations, thereof. The most preferred extracting solvents (extractant) for the extraction step of the present invention are methanol, ethanol, n-propanol, isopropanol or acetone or any mixtures respective combinations of said solvents, each in mixture with water. The use of pure organic solvents is not advantageous, due to the co-extraction of triglycerides.

The mixing ratio of water to the organic solvent, preferably water to the alcohol or water to acetone, in the extracting solvent is in a range of 10:90 to 90:10 (v/v), preferably in a range of 20:80 to 80:20 (v/v) and most preferably in a range of 30:70 to 70:30 (v/v), based in each case on the resulting extracting solvent.

Particularly preferred extracting solvents (extractants) are: methanol/water (3:7), methanol/water (1:1), methanol/water (7:3), ethanol/water (3:7), ethanol/water (1:1), ethanol/water (1:4), ethanol/water (7:3), isopropanol/water (3:7), isopropanol/water (1:1), isopropanol/water (7:3), acetone/water (3:7), acetone/water (1:1), acetone/water (7:3).

In order to improve the extraction yield, the oat source is extracted at a temperature ranging from 30° C. to 80° C., preferably from 40° C. to 70° C. and more preferably from 50° C. to 60° C. The extraction yield for milled oat grains increases with increasing temperatures between 40° C. and 70° C.

In a preferred variant, the oat beta-glucan extract is obtained by extraction under mild alkaline conditions, protein selection, ultrafiltration, double precipitation in ethanol and concentration of the oat beta-glucan. The state of the art describes extraction methodologies for oat beta-glucans based on solubility in hot water and alkaline solutions. Purification can be achieved for example by separating the co-extracted proteins by isoelectric precipitation, and following precipitating the beta-glucan using ammonium sulphate, 2-propanol or ethanol. Water, glycerin, 1,2-hexanediol and caprylyl glycol are optionally added to the thus obtained beta-glucan extract and standardised to a 1% by weight solution.

Another method to extract and purify cereal beta-glucan is described in EP 2 517 717.

Other methodologies for oat beta-glucans are based on extraction with an aqueous alcohol or acetone solution and subsequent chromatographic enrichment of the beta-glucan.

The oat extract according to the first aspect of the present invention, obtained by the methods described above, comprises at least one beta-glucan selected from the group consisting of a glucan with a beta (1→3) linked glucopyranosyl backbone, or a glucan with a beta (1→4) linked glucopyranoyl backbone, or a glucan with a mixed beta (1→3) (1→4) linked glucopyranosyl backbone.

In another variant, the oat extract comprises a mixture of two or three beta-glucans selected from the group consisting of a glucan with a beta (1→3) linked glucopyranosyl backbone, or a glucan with a beta (1→4) linked glucopyranoyl backbone, or a glucan with a mixed beta (1→3) (1→4) linked glucopyranosyl backbone.

In the composition according to the invention, the cereal beta-glucan is preferably an oat beta-glucan, more preferably a beta-glucan from oat extract, still more preferred wherein the cereal beta-glucan is selected from the group consisting of (1->3) beta-glucan, (1->4) beta-glucan, or a mixture of (1->3) beta-glucan and (1->4) beta-glucan.

The amount of the at least one β-glucan or the amount of the total beta-glucans present in the composition according to the present invention can be between 0.1 and 10.0% by weight, based on total weight of the composition. In a preferred variant, the concentration of the at least one beta-glucan or the total beta-glucans is 0.1 to 5.0% by weight, more preferred 0.8 to 1.5% by weight, and particularly preferred 0.9 to 1.2% by weight, based on the total weight of the composition.

The second main ingredient of the composition according to the first aspect of the present invention is at least one added inorganic and/or organic salt.

The term “added inorganic and/or organic salt” means, that the inorganic and/or salt is added actively to the composition according to the present invention and further means, that the salt is not yet integral part of the cereal beta-glucan or of the cereal extract comprising the at least one cereal beta-glucan.

The inorganic salt consists of a monovalent or divalent metal, and an anion of an inorganic acid. Preferably, the monovalent or divalent metal is an alkali metal cation or an alkaline earth metal cation. In a more preferred variant the metal cation is selected from the group consisting of Na⁺, K⁺, Ca⁺⁺, Mg⁺⁺ and Zn⁺⁺. Furthermore, the cation can be NH₄⁺. Preferably, the inorganic acid is selected from the group consisting of hydrohalic acid, sulfuric acid, phosphoric acid, and nitric acid. The inorganic salt can also be a mixture of two or more of the afore-mentioned inorganic salts.

The hydrohalic acid is favourably hydrochloric acid.

In a still more preferred variant of the present invention, the salt is selected from the group consisting of:
NaCl, KCl, CaCl₂), MgCl₂, ZnCl₂, NH₄Cl,
Na₂SO₄, K2SO₄, CaSO₄, MgSO₄, ZnSO₄,
Na₃PO₄, K3PO₄, Ca₃(PO₄)₂, Mg₃(PO₄)₂, Zn₃(PO₄)₂,
NaNO₃, KNO₃, Ca(NO₃)₂, Mg(NO₃)₂, Zn(NO₃)₂,
and a mixture of two or more of the afore-mentioned salts.

From the above inorganic salts, the chlorides are preferred at most, in particular NaCl, KCl, CaCl₂), MgCl₂ and NH₄Cl.

The NaCl salt has the most positive impact on stabilization and gelation of the liquid cereal beta-glucan comprising composition according to the present invention, and is, thus, particularly preferred. Advantageously, NaCl is easy to handle, cheap, common and well accepted and compatible in the preparation of foods, cosmetics or pharmaceutical preparations.

The organic salt consists of a monovalent or divalent metal, and an anion of an organic acid. Preferably, the monovalent or divalent metal is an alkali metal cation or an alkaline earth metal cation. In a more preferred variant, the metal cation is selected from the group consisting of Na⁺, K⁺, Ca⁺⁺, Mg⁺⁺ and Zn⁺⁺. Preferably, organic acid is selected from the group consisting of acetic acid, propionic acid, lactic acid, salicylic acid, succinic acid, malic acid, citric acid, gluconic acid, gluconolactone, sorbic acid, benzoic acid, and hyaluronic acid. The organic salt can also be a mixture of two or more of the afore-mentioned organic salts.

In a still more preferred variant of the present invention, the salt is selected from the group consisting of:
Na acetate, K acetate, Ca acetate, Mg acetate, Zn acetate,
Na propionate, K propionate, Ca propionate, Mg propionate, Zn propionate,
Na lactate, K lactate, Ca lactate, Mg lactate, Zn lactate,
Na salicylate, K salicylate, Ca salicylate, Mg salicylate, Zn salicylate,
Na succinate, K succinate, Ca succinate, Mg succinate, Zn succinate,
Na malate, K malate, Ca malate, Mg malate, Zn malate,
Na citrate, K citrate, Ca citrate, Mg citrate, Zn citrate,
Na gluconate, K gluconate, Ca gluconate, Mg gluconate, Zn gluconate, gluconolactone,
Na sorbate, K sorbate, Ca sorbate, Mg sorbate, Zn sorbate,
Na benzoate, K benzoate, Ca benzoate, Mg benzoate, Zn benzoate,
Na hyaluronate, K hyaluronate, Ca hyaluronate, Mg hyaluronate, Zn hyaluronate, and a mixture of two or more of said salts.

From the above organic salts, the citrates, sorbates and benzoates, as specified above, are the most potent salts, in particular Na citrate, K citrate, Na sorbate, K sorbate, Ca sorbate, Mg sorbate, Zn sorbate, Na benzoate, K benzoate, Ca benzoate, Mg benzoate, Zn benzoate.

Although the salt can be sodium benzoate, preferably, the salt is not Na benzoate. Instead K benzoate, Na sorbate and K sorbate are preferred.

In a still further preferred variant of the present invention, the added salt is the sum of the amount of the at least one added inorganic salt and/or the amount of at least one added organic salt.

The amount of the at least one added inorganic and/or organic salt or the total amount of added inorganic and/or organic salts present in the composition according to the present invention can be between 0.1 and 10.0% by weight, based on total weight of the composition. In a preferred variant, the concentration of the at least one added inorganic and/or organic salt or the total amount of added inorganic and/or organic salts is 0.5 to 4.0% by weight, still more preferred is 2.0 to 3.0% by weight, based on the total weight of the composition.

If the at least one cereal beta-glucan or the cereal extract comprising at least one cereal beta-glucan naturally comprises an inorganic and/or organic salt, the total salt content of the liquid composition of the present invention is accordingly higher than the concentration ranges of the added inorganic and/or organic salt as defined above. Accordingly, the salt content in the liquid composition of the present invention is the sum of the naturally occurring salt(s) content immanent to the cereal beta-glucan or the cereal comprising the beta-glucan plus the content of the added inorganic and/or organic salt(s).

The concentration of the at least one added inorganic and/or organic salt in the composition is selected or adjusted in such a way that the weight ratio of the at least one added inorganic and/or organic salt to the at least one beta-glucan or the total beta-glucans is in a range of 0.5:1 to 10:1. In a preferred variant, the composition comprises the at least one added inorganic and/or organic salt to the at least one beta-glucan or the total beta-glucans at a weight ratio in a range of 2:1 to 4:1. A most preferred composition is one in which the weight ratio of the at least one added inorganic and/or organic salt to the at least one beta-glucan or all the beta-glucans is in a ratio of 2:1.

In a more preferred variant, in the composition the total amount of the added salt is as much as high the total amount of beta-glucans, still more preferred the total amount of the added salt is 2 times higher than the total amount of beta-glucans.

In another preferred variant of the present invention according to the first aspect, the liquid composition according to the present invention comprises or consists of:
0.1 to 10.0% by weight, preferably 0.8 to 1.5% by weight, of at least one cereal beta-glucan or a cereal extract comprising at least one cereal beta-glucan; and
0.1 to 10% by weight, preferably 0.5 to 4.0% by weight, of at least one added inorganic and/or organic salt or a mixture thereof.
   based on the total weight of the composition.

The composition according to the first aspect of the present invention, as described above, exhibits an excellent stability. Additionally, the composition according to the present invention is characterized by a retarded gelation.

A stabilized beta-glucan solution means a liquid beta-glucan composition, which does not change its appearance and viscosity in the course of the time, in particular during storage. A stabilized beta-glucan solution further means a liquid beta-glucan composition, which displays less gelation than a control solution which has not been treated with an inorganic and/or organic salt as described above.

Gelation means the formation of a gel from a system with polymers. Branched polymers, i.e. in the present case the beta-glucan, can form links between the chains, which lead to progressively larger polymers. As the linking continues, larger branched polymers are obtained and at a certain extent of the reaction links between the polymer result in the formation of a single macroscopic molecule. At that point in the reaction, which is defined as gel point, the system loses fluidity and viscosity becomes very large. The onset of gelation, or gel point, is accompanied by a sudden increase in viscosity. Thus, for purposes of the present invention gelation is the process of forming a colloid in which the dispersed phase (beta-glucan) is combined with the continuous phase (water or aqueous solvent) to produce a viscous gel-like product.

By retarding gelation is meant a lowering of gel formation in the solution treated according to the present invention, i.e. by addition of an inorganic and/or organic salt, as compared to gelation exhibited by a control solution which has not been treated with an added inorganic and/or organic salt.

The composition according to the present invention exhibits improved stability with regard to viscosity and appearance and exhibits a retarded gelation upon cooling or in the course of storage, due to the addition of an inorganic and/or organic salt. Because of these beneficial properties, the composition according to the present invention can be provided in an easily storable liquid which is stable and easy to use and dose, as demonstrated in the examples below.

Preferably, the inventive composition is a solution, in particular wherein the maximum viscosity of the solution is from 100 to 50000 mPa·s, in particular 500 to 8000 to mPa·s, and/or in particular wherein the pH value of the solution is from 3.0 to 7.0, in particular 4.0 to 6.0.

Surprisingly, by adding an inorganic and/or organic salt to the dispersion or solution comprising or consisting of at least one cereal beta-glucan or a cereal extract comprising at least one cereal beta-glucan the stability with regard to viscosity and appearance of said liquid composition can be significantly improved. In addition, by adding an inorganic and/or organic salt to the liquid composition, gelation upon cooling and even during storage can considerably retarded, compared to a control solution which has not been stabilized with an inorganic and/or organic salt as described above, as this is demonstrated in the following examples.

Hence, the resulting liquid composition comprising the cereal beta-glucan or a cereal extract comprising a cereal beta-glucan according to the present invention is stable and can be stored without appreciable gelation, precipitation or deterioration of the product quality, compared to a composition which has not been treated with an inorganic and/or organic salt as described herein. Additionally, without gelation the composition provides a clear solution which is favourably for use in consumer products. Central to the present invention is thus the discovery of a simple and efficient method for producing stable liquid compositions of beta-glucan. The liquid composition typically displays deferred gelation as compared to untreated counterparts.

As it is obvious from the following Examples, inorganic and/or organic salts, as defined above, have a positive impact to retard the gelation time, i.e. prolong the time until gelation occurs, in terms of number of freeze/thaw cycles. The beta-glucan composition comprising an added salt such as NaCl, KCl or CaCl₂) (samples 14, 15 or 16) have a higher number of freeze/thaw cycles, compared to a control solution which has not been stabilized with an inorganic and/or organic salt (sample 13).

In particular, the best performance has NaCl. As it is obvious from the results in Example 3 and from Table 5, the addition of NaCl to a sample of beta-glucan solution considerably improves the gelation delay, i.e. the sample withstands 11 freeze/thaw cycles until appreciable gelation occurs, whereas the same sample of beta-glucan solution without the addition of NaCl withstands only 5 freeze/thaw cycles, until gelation occurs. Hence, the combination of cereal beta-glucan or a cereal extract comprising at least one cereal beta-glucan with NaCl is particularly preferred. Advantageously, NaCl is easy to handle, cheap, common and well accepted and compatible in the preparation of foods, cosmetics or pharmaceutical preparations. The addition of NaCl thus beneficial allows a simple and efficient method for producing stable solutions of beta-glucan and retards the formation of gels as compared to untreated counterparts.

Without bonded to any theory, it is assumed that by adding an inorganic and/or organic salt to the cereal beta-glucan comprising composition, the electrostatic repulsion prevents from chains association.

In a still preferred variant the liquid composition according to the present invention further comprises optionally at least one multifunctional compound. Multifunctional compounds are compounds or substances that enhance the performance of active ingredients, improves formula aesthetics including dispensability of pigments, stabilizes emulsions, act as solubilizer or moisturizer and also provide product protection. These multitaskers help to eliminate the overall number of ingredients while maximizing formula benefits and optimizing consumer experience.

Multitaskers or multifunctional compounds in the context of the present invention are Na benzoate, Na hyaluronate, polyols respective glycols, such as 1,2-propanediol (propylene glycol), 1,3-propanediol, 1,2-butanediol, 1,3-butanediol (butylene glycol), 1,2-pentanediol (pentylene glycol; Hydrolite® 5; green version or any grade), 1,2-hexanediol (Hydrolite® 6), 1,2-octanediol (caprylyl glycol; Hydrolite® 8), 1,2-decanediol (decylene glycol), or glycerol, phenoxyethanol, ethylhexylglycerin, glyceryl caprylate, hydroxyacetophenone, methylbenzyl alcohol, o-cymen-5-ol, benzyl alcohol, tropolone, or mixtures of two or more of said multitaskers, which are known compound commonly used as ingredient in cosmetics or pharmaceutical preparations.

From the above multifunctional ingredients, glycols that for example boosts the performance of active ingredients, moisturizes the skin, improves the sensorial profile of formulas and enhances product protection, are particularly preferred.

Preferably, the polyol respective glycol added to the liquid beta-glucan composition according to the present invention is selected from the group consisting of 1,2-pentanediol (pentylen glycol; Hydrolite® 5; green version or any grade), 1,2-hexanediol (Hydrolite® 6), and 1,2-octanediol (caprylyl glycol; Hydrolite® 8).

Surprisingly, the addition of any glycol, preferably 1,2-pentanediol (pentylene glycol; Hydrolite® 5; green version or any grade), to the liquid composition according to the present invention further improves gelation retard. Pentylene glycol as such has no positive impact on stabilization and gelation of a liquid cereal beta-glucan comprising composition.

Surprisingly, the combined use of 1,2-pentanediol (pentylene glycol (Hydrolite® 5; green) with an inorganic or organic salt has the most positive impact on stabilization and gelation of the liquid cereal beta-glucan comprising composition, and is, thus, particularly preferred. The combined use of NaCl at 2% by weight with Hydrolite® 5 green in a beta-glucan composition definitely shows superior performance: As can be seen from Table 6, the addition of Hydrolite® 5 green to the cereal beta-glucan comprising composition according to the present invention (samples 14 (comprising NaCl), 15 (comprising CaCl₂) dihydrate), 16 (comprising KCl) and 17 (comprising Na benzoate) results in a superior and synergistically performing beta-glucan composition having a high number of freeze/thaw cycles, compared to a beta-glucan control solution (sample 13) which comprises only Hydrolite® 5 green but has not been stabilized with an inorganic and/or organic salt.

Hence, the additional use of 1,2-pentanediol (pentylene glycol (Hydrolite® 5; green) thus beneficial allows to further improve stability and gelation retard of cereal beta-glucan comprising compositions as compared to untreated counterparts.

The amount of the at least one added multifunctional compound or the total amount of added multifunctional compounds present in the composition according to the present invention can be between 0.1 and 10.0% by weight, based on total weight of the composition. In a preferred variant, the concentration of the at least one added multifunctional compound or the total amount of added multifunctional compounds is 1.0 to 6.0% by weight, still more preferred is 1.0 to 3.0% by weight, based on the total weight of the composition.

In a second aspect, the present invention relates to a method for producing a liquid cereal beta-glucan composition having retarded gelation, wherein said method comprises:
(a) providing a solution comprising or consisting of a at least one cereal beta-glucan or a cereal extract comprising at least one cereal beta-glucan;
(b) adding to the solution of step (a) at least one inorganic and/or organic salt or a mixture thereof to obtain a mixture;
(c) optionally adjusting the pH value of the mixture to a pH value in a range from 3.0 to 7.0, in particular in a range from 4.0 to 6.0; and
(d) optionally heating said mixture to a temperature in a range from 20° C. to 100° C., preferably in a range from 50° C. to 80° C.

In the first step of the method according to the second aspect of the present invention, a solution comprising or consisting of at least one cereal beta-glucan as described above or a cereal extract comprising at least one cereal beta-glucan as described above is provided.

With regard to the cereal beta-glucan or the cereal extract comprising the beta-glucan, reference is made to the detailed description above for the composition according to the first aspect of the present invention is made. The same definitions, preferred variants, modifications, properties and beneficial effects as described are applicable to the method according to the second aspect of the present invention.

In the method according to the second aspect of the present invention, the cereal beta-glucan is preferably an oat beta-glucan, in particular a beta-glucan from oat extract, in particular wherein the beta-glucan is selected from the group consisting of (1->3) beta-glucan, (1->4) beta-glucan, or a mixture of (1->3) beta-glucan and (1->4) beta-glucan.

Preferably, said composition is employed as aqueous dispersion or solution. An aqueous dispersion or solution in the context of the present invention means, that the cereal beta-glucan or a cereal extract comprising at least one cereal beta-glucan is dispersed or dissolved in water or an aqueous solution. Preferably, the aqueous solvent for preparing the aqueous solution is selected from the group consisting of aqueous/alcoholic solvents, preferably aqueous ethanolic solvents, glycerine, and propylene glycol.

The amount of the at least one beta-glucan or the amount of the total beta-glucans used in the method according to the present invention is between 0.1 and % by weight, based on total weight of the composition. In a preferred variant, the at least one beta-glucan or the total beta-glucans is used in a concentration of 0.1 to % by weight, more preferred in a concentration of 0.8 to 1.5% by weight, and particularly preferred in a concentration of 0.9 to 1.2% by weight, based on the total weight of the composition.

In a further step (b), at least one inorganic and/or organic salt or a mixture thereof is added to the dispersion or solution from step (a).

The inorganic salt used in the method according to the present invention consists of a monovalent or divalent metal, and an anion of an inorganic acid. Preferably, the monovalent or divalent metal is an alkali metal cation or an alkaline earth metal cation. In a more preferred variant the metal cation is selected from the group consisting of Na⁺, K⁺, Ca⁺⁺, Mg⁺⁺ and Zn⁺⁺. Furthermore, the cation may be NH₄⁺. Preferably, the inorganic acid is selected from the group consisting of hydrohalic acid, sulfuric acid, phosphoric acid, and nitric acid. The inorganic salt can also be a mixture of two or more of the afore-mentioned inorganic salts.

The hydrohalic acid is favourably hydrochloric acid.

In a still more preferred variant of the present invention, the salt is selected from the group consisting of:
NaCl, KCl, CaCl₂), MgCl₂, ZnCl₂, NH₄Cl,
Na₂SO₄, K2SO₄, CaSO₄, MgSO₄, ZnSO₄,
Na₃PO₄, K3PO₄, Ca₃(PO₄)₂, Mg₃(PO₄)₂, Zn₃(PO₄)₂,
NaNO₃, KNO₃, Ca(NO₃)₂, Mg(NO₃)₂, Zn(NO₃)₂,
and a mixture of two or more of the afore-mentioned salts.

From the above inorganic salts, the chlorides are preferred at most, in particular NaCl, KCl, CaCl₂), MgCl₂ and NH₄Cl.

The NaCl salt has the most positive impact on stabilization and gelation of the composition, and is, thus, particularly preferred. Advantageously, NaCl is easy to handle, cheap, common and well accepted and compatible in the preparation of foods, cosmetics or pharmaceutical preparations.

The organic salt used in the method according to the present invention consists of a monovalent or divalent metal, and an anion of an organic acid. Preferably, the monovalent or divalent metal is an alkali metal cation or an alkaline earth metal cation. In a more preferred variant the metal cation is selected from the group consisting of Na⁺, K⁺, Ca⁺⁺, Mg⁺⁺ and Zn⁺⁺. Preferably, the anion stems from an organic acid. In a more preferred variant the organic acid is selected from the group consisting of acetic acid, propionic acid, lactic acid, salicylic acid, succinic acid, malic acid, citric acid, gluconic acid, gluconolactone, sorbic acid, benzoic acid, and hyaluronic acid. The organic salt can also be a mixture of two or more of the afore-mentioned organic salts.

In a still more preferred variant of the present invention, the salt is selected from the group consisting of
Na acetate, K acetate, Ca acetate, Mg acetate, Zn acetate,
Na propionate, K propionate, Ca propionate, Mg propionate, Zn propionate,
Na lactate, K lactate, Ca lactate, Mg lactate, Zn lactate,
Na salicylate, K salicylate, Ca salicylate, Mg salicylate, Zn salicylate,
Na succinate, K succinate, Ca succinate, Mg succinate, Zn succinate,
Na malate, K malate, Ca malate, Mg malate, Zn malate,
Na citrate, K citrate, Ca citrate, Mg citrate, Zn citrate,
Na gluconate, K gluconate, Ca gluconate, Mg gluconate, Zn gluconate, gluconolactone,
Na sorbate, K sorbate, Ca sorbate, Mg sorbate, Zn sorbate,
Na benzoate, K benzoate, Ca benzoate, Mg benzoate, Zn benzoate,
Na hyaluronate, K hyaluronate, Ca hyaluronate, Mg hyaluronate, Zn hyaluronate,
and a mixture of two or more of said salts.

From the above organic salts, the citrates, sorbates and benzoates, as specified above, are the most potent salts, in particular Na citrate, K citrate, Na sorbate, K sorbate, Ca sorbate, Mg sorbate, Zn sorbate, Na benzoate, K benzoate, Ca benzoate, Mg benzoate, Zn benzoate.

Although the salt can be sodium benzoate, preferably, the salt is not Na benzoate. Instead K benzoate, Na sorbate and K sorbate are preferred.

In a still further preferred variant of the method according to the present invention, the added salt is a combination of at least one inorganic salt and/or of at least on organic salt.

The inorganic and/or organic salt is added to the cereal beta-glucan comprising dispersion or solution either as solid or as an aqueous solution, preferably dissolved in water.

The amount of the at least one added inorganic and/or organic salt or the total amount of added inorganic and/or organic salts used in the method according to the present invention is between 0.1 and 10.0% by weight, based on total weight of the composition. In a preferred variant, the concentration of the at least one added inorganic and/or organic salt or the total amount of added inorganic and/or organic salts is 0.5 to 4.0% by weight, still more preferred is 2.0 to 3.0% by weight, based on the total weight of the composition.

The beta-glucan dispersion or solution employed in step (a) of the method according to the present invention usually has an initial pH value in a range from 3.0 to 7.0. In order to further improve the stability and retard of the gelation of the cereal beta-glucan comprising composition, the pH value of the mixture is optionally adjusted to a pH value in a range from 3.0 to 7.0. Preferably, the pH value is adjusted to a pH value in a range from 4.0 to 6.0, as this is demonstrated in the following examples.

If the initial pH value differs from the optimum pH value in a range of 3.0 to 7.0, the pH value is adjusted to the above pH value range by addition of an organic acid, preferably citric acid or lactic acid.

In a particular preferred variant, the method according to the second aspect of the present invention optionally comprises as a further optional step the heating of the said mixture to a temperature in a range from 20° C. to 100° C. Preferably, the mixture is heated to a temperature in a range from 20° C. to 90° C. Still more preferred is a temperature in a range from 20° C. to 80° C. and particularly preferred is a temperature in a range from 50° C. to 80° C.

Surprisingly, the additional heating treatment of said mixture comprising the beta-glucan and the inorganic and/or organic salt further improves the stability with regard to viscosity and appearance of the solution and the retard of gelation upon cooling or during storage of the composition.

In a third aspect the present invention relates to a method for transforming a gelled cereal beta-glucan comprising or consisting composition, said method comprising:
(i) providing a gelled composition comprising or consisting of a at least one cereal beta-glucan or a cereal extract comprising at least one cereal beta-glucan;
(ii) heating said gelled composition to a temperature in a range from 20° C. to 100° C. to obtain a solution, preferably in a range from 50° C. to 80° C.;
(iii) adding to the solution of step (ii) at least one inorganic and/or organic salt or mixtures thereof to obtain a mixture; and
(iv) optionally adjusting the pH value of the mixture to a pH value in a range from 3.0 to 7.0, preferably in a range from 4.0 to 6.0.

The method according to the third aspect of the present invention serves to transform a cereal beta-glucan comprising composition where gelation has already occurred, into a liquid cereal beta-glucan comprising composition that can be used directly or stored for future use.

In the first step of the method according to the third aspect of the present invention, an already gelled composition comprising or consisting of at least one cereal beta-glucan as described above or a cereal extract comprising at least one cereal beta-glucan as described above is provided.

With regard to the cereal beta-glucan or the cereal extract comprising the beta-glucan, reference is made to the detailed description above for the composition according to the first aspect of the present invention is made. The same definitions, preferred variants, modifications, properties and beneficial effects as described are applicable to the method according to third aspect of the present invention.

In the method according to the second aspect of the present invention, the cereal beta-glucan is preferably an oat beta-glucan, in particular a beta-glucan from oat extract, in particular wherein the beta-glucan is selected from the group consisting of (1->3) beta-glucan, (1->4) beta-glucan, or a mixture of (1->3) beta-glucan and (1->4) beta-glucan.

The amount of the at least one beta-glucan or the amount of the total beta-glucans used in the method according to the present invention is between 0.1 and % by weight, based on total weight of the composition. In a preferred variant, the at least one beta-glucan or the total beta-glucans is used in a concentration of 0.1 to % by weight, more preferred in a concentration of 0.8 to 1.5% by weight, still more preferred is 0.9 to 1.2% by weight, based on the total weight of the composition.

In a further step, the gelled composition is heated to a temperature in the range of from 20° C. to 100° C., preferably to a temperature in the range of from 40° C. to 90° C., more preferably to a temperature in the range of from 50° C. to 80° C., and particularly preferably to a temperature in the range of from 60° C. to 70° C., to obtain a solution. The heat breaks the gel.

In order to further improve the stability and retard of the gelation of the cereal beta-glucan comprising composition, in a next step at least one inorganic and/or organic salt or a mixture thereof is added to the solution of step (ii).

With regard to the inorganic and/or organic salt, reference is made to the detailed description above for the composition according to the first aspect or to the detailed description above for the method according to the second aspect, in particular step (b). The same definitions, preferred variants, modifications, properties and beneficial effects as described are applicable to the method according to the third aspect of the present invention.

While heat breaks the gel, surprisingly, the adding of an inorganic and/or organic salt to the gelled solution comprising or consisting of at least one cereal beta-glucan or a cereal extract comprising at least one cereal beta-glucan, improves the aspect of transforming the gelled solution into a stable, less jelly, aqueous beta-glucan solution.

In order to further improve the stability and retard of the gelation of the cereal beta-glucan comprising composition, the pH value of the mixture obtained in step (iii) is optionally adjusted to a pH value in a range from 3.0 to 7.0. Preferably, the pH value is adjusted to a pH value in a range from 4.0 to 6.0, as this is demonstrated in the following examples. If the pH value of the cereal beta-glucan comprising composition differs from the optimum pH value in a range of 3.0 to 7.0, the pH value is adjusted to the above pH value by addition of an organic acid, preferably citric acid or lactic acid.

The method according to the second aspect or the third aspect of the present invention results in a cereal beta-glucan comprising composition which can be used directly or stored for future use. The composition is characterized by an improved stability regarding viscosity and appearance. In particular, the beta-glucan composition is distinguished by a retarded gelation upon cooling and during storage.

In a further aspect of the present invention, a liquid beta-glucan composition comprising a cereal beta-glucan or a cereal extract comprising a cereal beta-glucan is provided which is obtainable using the method according to the second aspect or third aspect of the present invention.

As demonstrated by the following examples, the composition according to the present invention can be provided in a stable, easily storable liquid, where gelation is retarded and which is easy to use and dose. Additionally, without gelation the composition provides a clear solution which is advantageously for the use in consumer products.

The composition according to the present invention or obtained by a method according to the present invention, can be used as such or in the preparation of food, food supplements, cosmetic, pharmaceutical or veterinary preparations.

Due to the presence of at least one beta-glucan and its enhanced anti-oxidant, anti-inflammatory, anti-itching, anti-irritant and anti-atherogenic activities, the composition according to the present invention can be favourably used as a dermatological cosmetic or as a medicament in the prevention and treatment of dermatological disorders. Due to their anti-oxidant and strong cholesterol and triglyceride lowering properties of beta-glucan, which is known for over two decades, the composition according to the present invention, can also be favourably used in the prevention and/or treatment of dermatological diseases associated with increased ROS production or prevention and/or treatment of cardiovascular diseases and of allergic reactions.

A further aspect of the present invention therefore relates to the use of the composition according to the first aspect of the present invention or which is obtained using the method according to the present invention as a cosmetic.

Because of its improved beneficial biological activities and properties, due to the presence of beta-glucan, the composition according to the first aspect of the present invention is beneficially suitable in skin care, scalp care, hair care, nail care or in the prevention and/or treatment of skin conditions, intolerant and sensitive skin, skin irritation, skin reddening, wheals, pruritus (itching), skin aging, wrinkle formation, loss of skin volume, loss of skin elasticity, pigment spots, pigment abnormalities, or dry skin, i.e. for moisturising the skin.

A further aspect of the present invention therefore relates to the use of the composition according to the first aspect of the present invention or which is obtained using the method according to the present invention as a medicament.

In particular, the composition according to the first aspect of the present invention is beneficially suitable for use in the prevention and/or treatment of dermatological or keratological diseases, preferably of dermatological or keratological diseases having a barrier related, inflammatory, immunoallergic, atherogenic, xerotic or hyperproliferative type component or in the prevention and/or treatment of dermatological diseases associated with increased ROS production or in the prevention and/or treatment of cardiovascular diseases, allergic reactions, coronary heart disease, for decreasing the level of LDL cholesterol and lipids in blood serum, for reducing blood pressure and for improving sensitivity to insulin and for enabling the control of blood glucose levels.

The dermatological or keratological disorders are selected from the group consisting of eczema, psoriasis, seborrhea, dermatitis, erythema, pruritus (itching), otitis, inflammation, irritation, fibrosis, lichen planus, pityriasis rosea pityriasis versicolor, autoimmune bullous diseases, urticarial, angiodermal and allergic skin reactions, and wound healing, and/or the skin diseases associated with increased ROS production are selected from the group consisting of atopic dermatitis, neurodermitis, psoriasis, rosacea, acneiform eruptions, sebostasis and xerosis.

The use of the composition for these respective purposes corresponds to a method for imparting the respective therapeutic activity to a substance by adding a therapeutically effective amount of the composition or oat extract.

Within the context of the present invention, an effective amount of a composition is the amount of each active component that is sufficient to show a benefit, such as a reduction in a symptom associated with the disorder, disease or condition to be treated. When applied to a combination, as in the present case, the term refers to the amount of the combined active ingredients resulting in the benefit.

Due to its marked radical-scavenging activity, and therefore anti-oxidative effect, due to the presence of beta-glucan, the composition according to the first aspect of the present invention is also beneficially suitable for the preparation of foods, food supplements or veterinary products.

The composition according to the first aspect of the present invention can be either used as such, or can be easily incorporated into conventional food, food supplements, cosmetic, pharmaceutical or veterinary preparations.

Accordingly, in a further aspect the present invention relates to food, food supplements, cosmetic, pharmaceutical or veterinary preparations which comprise the composition according to the present invention or which is obtained using the method according to the present invention. In a preferred variant of the present invention, a functional food which includes the composition according to the present invention is provided as an effective ingredient for preventing or ameliorating the above disorders.

In a preferred variant, the food, food supplements, cosmetic, pharmaceutical or veterinary preparations comprise the composition according to the present invention or which is obtained using the method according to the present invention in an amount of 0.1 to 100.0% by weight, more preferably in an amount of 1.0 to 20% by weight, more preferred 1 to 10% by weight, most preferred 1 to 5% by weight, based on the total weight of the preparation. Especially, a cosmetic preparation or a food supplement contains the composition according to the present invention in an amount up to 100% by weight, preferably in an amount of 1.0 to 10% by weight, based on the total weight of the preparation.

Within the context of the present invention, it is also possible—and in some cases advantageous—to combine the composition or the food, food supplement, cosmetic, pharmaceutical or veterinary preparation, according to the present invention with other active agents or additives.

Optionally, other conventional cosmetically and/or dermatologically active substances as further described below, may be added to the composition according to the invention in order to obtain a ready-for-use preparation such as food, food supplements, cosmetic, pharmaceutical or veterinary preparations, for the sake of convenience hereinafter designated as “preparation”. The term “preparation” means a preparation comprising the liquid cereal beta-glucan composition according to the present invention.

The beta-glucan composition or the preparation, comprising the beta-glucan composition, preferably a cosmetic or pharmaceutical preparation, according to the present invention can advantageously be combined with other cosmetically or pharmaceutically active agents and/or additives or auxiliaries, such as are customarily used in such preparations, such as for example antioxidants, perfume oils, anti-foaming agents, colorants, pigments having a colouring action, thickeners, surface-active substances, emulsifiers, plasticising substances, moistening and/or moisture-retaining substances, fats, oils, waxes or other conventional constituents of a cosmetic or pharmaceutical preparation, such as alcohols, polyols, polymers, foam stabilisers, electrolytes, organic solvents or silicone derivatives. Any conceivable antioxidants, perfume oils, anti-foaming agents, colorants, pigments having a colouring action, thickeners, surface-active substances, emulsifiers, plasticising substances, moistening and/or moisture-retaining substances, fats, oils, waxes, alcohols, polyols, polymers, foam stabilisers, electrolytes, organic solvents or silicone derivatives that are suitable or customary in cosmetic or pharmaceutical applications, and/or cosmetically or pharmaceutically acceptable excipients, as in detail described and exemplified below.

Since dermatological conditions or diseases are often associated with dry skin, scratched skin, skin lesions or even inflammation, the beta-glucan composition or the preparation, comprising the beta-glucan composition, preferably a cosmetic or pharmaceutical preparation, according to the present invention can particularly advantageously contain preferably anti-inflammatories, antibacterial or antimycotic substances, substances having a reddening-alleviating or itch-alleviating action, lenitive substances, anti-dandruff, moisturizers and/or cooling agents, osmolytes, keratolytic substances, nurturing substances, anti-inflammatory, antibacterial or antimycotic substances, substances having a reddening-alleviating or itch-alleviating action, lenitive substances, antidandruff substances, or other active compounds such as solvents, fragrances, antioxidants, preservatives, (metal) chelating agents, penetration enhancers, and mixtures thereof.

Itching occurs with particular intensity when the skin is dry. The use of skin-moisturising and/or moisture-retaining substances can significantly alleviate itching. The beta-glucan composition or the preparation, comprising the beta-glucan composition, preferably a cosmetic or pharmaceutical preparation, according to the present invention can therefore advantageously also contain the following moisturising and/or moisture-retaining substances: sodium lactate, urea, urea derivatives, alcohols, glycerol, diols such as propylene glycol, hexylene glycol, 1,2-pentanediol, 1,2-hexanediol, 1,2-heptanediol, 1,2-octanediol, 1,2-nonanediol, 1,2-decanediol or mixtures of said diols, in particular mixtures of 1,2-hexanediol and 1,2-octanediol, collagen, elastin or hyaluronic acid, diacyl adipates, petrolatum, urocanic acid, lecithin, panthenol, phytantriol, lycopene, (pseudo-)ceramides, glycosphingolipids, cholesterol, phytosterols, chitosan, chondroitin sulphate, lanolin, lanolin esters, amino acids, alpha-hydroxy acids (such as citric acid, lactic acid, malic acid) and their derivatives, mono-, di- and oligosaccharides such as glucose, galactose, fructose, mannose, fructose and lactose, polysugars such as R-glucans, in particular 1,3-1,4-β-glucan from oats, alpha-hydroxy fatty acids, triterpene acids such as betulinic acid or ursolic acid, and algae extracts.

The use of cooling agents can alleviate itching. The beta-glucan composition or the preparation, comprising the beta-glucan composition, preferably a cosmetic or pharmaceutical preparation, according to the present invention can therefore advantageously also contain one or more cooling agent(s). Preferred individual cooling agents for use within the framework of the present invention are listed below. The person skilled in the art can add many other cooling agents to this list; the cooling agents listed can also be used in combination with one another: 1-menthol, d-menthol, racemic menthol, menthone glycerol acetal (trade name: Frescolat® MGA), menthyl lactate (trade name: Frescolat® ML; menthyl lactate is preferably I-menthyl lactate, in particular 1-menthyl 1-lactate), substituted menthyl-3-carboxamides (such as menthyl-3-carboxylic acid N-ethyl amide), 2-isopropyl-N-2,3-trimethyl butanamide, substituted cyclohexane carboxamides, 3-menthoxypropane-1,2-diol, 2-hydroxyethyl menthyl carbonate, 2-hydroxypropyl menthyl carbonate, N-acetylglycine menthyl ester, isopulegol, hydroxycarboxylic acid menthyl esters (such as menthyl 3-hydroxybutyrate), monomenthyl succinate, 2-mercaptocyclodecanone, menthyl 2-pyrrolidin-5-one carboxylate, 2,3-dihydroxy-p-menthane, 3,3,5-trimethyl cyclohexanone glycerol ketal, 3-menthyl-3,6-di- and trioxaalkanoates, 3-menthyl methoxyacetate and icilin.

Cooling agents which are preferred due to their particular synergistic effect are 1-menthol, d-menthol, racemic menthol, menthone glycerol acetal (trade name: Frescolat® MGA), menthyl lactate (preferably 1-menthyl lactate, in particular 1-menthyl 1-lactate (trade name: Frescolat® ML)), substituted menthyl-3-carboxamides (such as menthyl-3-carboxylic acid N-ethyl amide), 2-isopropyl-N-2,3-trimethyl butanamide, substituted cyclohexane carboxamides, 3-menthoxypropane-1,2-diol, 2-hydroxyethyl menthyl carbonate, 2-hydroxypropyl menthyl carbonate and isopulegol.

Particularly preferred cooling agents are 1-menthol, racemic menthol, menthone glycerol acetal (trade name: Frescolat® MGA), menthyl lactate (preferably 1-menthyl lactate, in particular 1-menthyl 1-lactate (trade name: Frescolat® ML)), 3-menthoxypropane-1,2-diol, 2-hydroxyethyl menthyl carbonate and 2-hydroxypropyl menthyl carbonate. Very particularly preferred cooling agents are 1-menthol, menthone glycerol acetal (trade name: Frescolat® MGA) and menthyl lactate (preferably 1-menthyl lactate, in particular 1-menthyl 1-lactate (trade name: Frescolat® ML).

The beta-glucan composition or the preparation, comprising the beta-glucan composition, preferably a cosmetic or pharmaceutical preparation, according to the present invention can also contain one or more osmolyte(s). Examples of osmolytes which may be mentioned here include substances from the group comprising sugar alcohols (myoinositol, mannitol, sorbitol), quaternary amines such as taurine, choline, betaine, betaine glycine, ectoine, diglycerol phosphate, phosphorylcholine or glycerophosphorylcholines, amino acids such as glutamine, glycine, alanine, glutamate, aspartate or proline, phosphatidylcholine, phosphatidylinositol, inorganic phosphates, and polymers of said compounds, such as proteins, peptides, polyamino acids and polyols. All osmolytes simultaneously have a skin-moisturising action.

Preferably, keratolytic substances can also be used in the beta-glucan composition or the preparation, comprising the beta-glucan composition, according to the present invention. Keratolytic compounds include the large group of alpha-hydroxy acids. Salicylic acid is for example preferably used.

In the beta-glucan composition or the preparation, comprising the beta-glucan composition, preferably a cosmetic or pharmaceutical preparation, according to the present invention a high proportion of in particular nurturing substances is also particularly advantageous because of the reduced trans-epidermal water loss due to lipophilic components. In one preferred embodiment, the cosmetic or pharmaceutical, in particular dermatological, preparation contain one or more nurturing animal and/or vegetable fats and oils such as olive oil, sunflower oil, refined soybean oil, palm oil, sesame oil, rapeseed oil, almond oil, borage oil, evening primrose oil, coconut oil, shea butter, jojoba oil, sperm oil, tallow, neatsfoot oil and lard, and optionally other nurturing components such as fatty alcohols having 8 to 30 C atoms. The fatty alcohols used here can be either saturated or unsaturated and either linear or branched. Nurturing substances which can be particularly preferably combined with the mixtures according to the present invention also include in particular ceramides, understood here to mean N-acylsphingosines (fatty acid amides of sphingosine) or synthetic analogues of such lipids (so-called pseudo-ceramides) which markedly improve the water retention capacity of the stratum corneum; phospholipids, such as soy lecithin, egg lecithin and cephalins; and petrolatum, paraffin oils and silicone oils, the latter including inter alia dialkyl- and alkylarylsiloxanes such as dimethylpolysiloxane and methylphenylpolysiloxane and their alkoxylated and quaternised derivatives.

The beta-glucan composition or the preparation, comprising the beta-glucan composition, preferably a cosmetic or pharmaceutical preparation, according to the present invention can also contain one or more anti-inflammatory substances. Advantageously, the anti-inflammatory active compounds are steroidal anti-inflammatory substances of the corticosteroid type, such as for example hydrocortisone, dexamethasone, dexamethasone phosphate, methylprednisolone or cortisone, wherein this list may be expanded by adding other steroidal anti-inflammatory agents. Non-steroidal anti-inflammatory agents can also be used, for example: oxicams, such as piroxicam or tenoxicam; salicylates, such as aspirin, Disalcid®, Solprin® or fendosal; acetic acid derivatives, such as diclofenac, fenclofenac, indomethacin, sulindac, tolmetin or clindanac; fenamates, such as mefenamic, meclofenamic, flufenamic or niflumic acid; propionic acid derivatives, such as ibuprofen, naproxen, benoxaprofen; or pyrazoles, such as phenylbutazone, oxyphenylbutazone, febrazone or azapropazone. Alternatively, natural anti-inflammatory substances can be used.

Other antibacterial or antimycotic active substances can also particularly advantageously be used in the beta-glucan composition or the preparation, comprising the beta-glucan composition, preferably a cosmetic or pharmaceutical preparation, according to the present invention, wherein any antibacterial or antimycotic active substances can be used which are suitable or customary in cosmetic or pharmaceutical, in particular dermatological applications. In addition to the large group of conventional antibiotics, other products which are advantageous here include those relevant to cosmetics such as in particular triclosan, climbazole, octoxyglycerin, Octopirox® (1-hydroxy-4-methyl-6-(2,4,4-trimethylpentyl)-2(1H)-pyridone 2-aminoethanol salt), chitosan, farnesol, glycerol monolaurate or combinations of said substances, which are used inter alia against underarm odour, foot odour or dandruff.

The beta-glucan composition or the preparation, comprising the beta-glucan composition, preferably a cosmetic or pharmaceutical preparation, according to the present invention can also contain one or more lenitive substances, wherein any lenitive substances can be used which are suitable or customary in cosmetic or pharmaceutical applications such as alpha-bisabolol, azulene, guaiazulene, 18-beta-glycyrrhetinic acid, allantoin, Aloe vera juice or gel, extracts of Hamamelis virginiana (witch hazel), Echinacea species, Centella asiatica, chamomile, Arnica monatana, Glycyrrhiza species, algae, seaweed and Calendula officinalis, and vegetable oils such as sweet almond oil, baobab oil, olive oil and panthenol, Laureth-9, Trideceth-9 and 4-t-butylcyclohexanol.

In addition, the beta-glucan composition or the preparation, comprising the beta-glucan composition, preferably a cosmetic or pharmaceutical preparation, according to the present invention can also particularly advantageously contain antidandruff substances, including triclosan, climbazole, octoxyglycerin, Octopirox® (1-hydroxy-4-methyl-6-(2,4,4-trimethylpentyl)-2(1H)-pyridone 2-aminoethanol salt), chitosan, farnesol, glycerol monolaurate, Propanediol Monocaprylate or combinations of said substances, which are used inter alia against underarm odour, foot odour or dandruff.

In addition, the beta-glucan composition or the preparation, comprising the beta-glucan composition, preferably a cosmetic or pharmaceutical preparation, according to the present invention can also particularly advantageously contain perspiration-inhibiting active compounds (antiperspirants) for controlling body odour. Perspiration-inhibiting active compounds used include in particular aluminium salts, such as aluminium chloride, chlorohydrate, nitrate, sulphate, acetate, etc. The use of zinc, magnesium or zirconium compounds can however also be advantageous. Aluminium salts and, to a somewhat lesser extent, aluminium/zirconium salt combinations have proven useful as cosmetic or pharmaceutical antiperspirants. Partially neutralised aluminium hydroxychlorides, which are therefore more tolerable to the skin but are not quite as effective, are also noteworthy. Substances other than aluminium salts can also be used, such as for example: (a) protein-precipitating substances such as inter alia formaldehyde, glutaraldehyde, natural and synthetic tanning agents and trichloroacetic acid, which cause surface closure of the sweat glands; (b) local anaesthetics, including dilute solutions of for example lidocaine, prilocaine or mixtures of the same, which switch off the sympathetic supply to the sweat glands by blocking the peripheral nerve paths; (c) zeolites of the X, A or Y type, which reduce sweat secretion and also act as adsorbents for bad odours; and (d) botulinus toxin (the toxin of the bacterium Chlostridium botulinum), which is also used in hyperhidrosis (pathological increase in sweat secretion), and the action of which is based on irreversibly blocking the release of the transmitter substance acetylcholine which is relevant to sweat secretion.

A combination with (metal) chelating agents can also be advantageous in the beta-glucan composition or the preparation, comprising the beta-glucan composition, preferably a cosmetic or pharmaceutical preparation, according to the present invention, wherein any metal chelating agents can be used which are suitable or customary in cosmetic or pharmaceutical applications. Preferred (metal) chelating agents include α-hydroxy fatty acids, phytic acid, lactoferrin, α-hydroxy acids, such as inter alia gluconic acid, glyceric acid, glycolic acid, isocitric acid, citric acid, lactic acid, malic acid, mandelic acid, tartaric acid, as well as humic acids, bile acids, bile extracts, bilirubin, biliverdin or EDTA, EGTA and their derivatives. The use of one or more chelating agent(s) improves the stability of the surfactant containing preparation according to the present invention.

The beta-glucan composition or the preparation, comprising the beta-glucan composition, preferably a cosmetic or pharmaceutical preparation, according to the present invention can advantageously be combined with substances that absorb UV radiation in the UVB range in order to provide cosmetic preparations that protect the hair and/or skin against the entire range of ultraviolet radiation. They can also serve as sunscreens for hair. If the preparations according to the present invention contain UVB filter substances, these can be oil-soluble or water-soluble. Advantageous oil-soluble UVB filters include: 3-benzylidene camphor derivatives, preferably 3-(4-methylbenzylidene)camphor, 3-benzylidenecamphor; 4-aminobenzoic acid derivatives, preferably 2-ethylhexyl 4-(dimethylamino)benzoate, amyl 4-(dimethylamino)benzoate; esters of cinnamic acid, preferably 2-ethylhexyl 4-methoxycinnamate, isopentyl 4-methoxycinnamate; esters of salicylic acid, preferably 2-ethylhexyl salicylate, 4-isopropylbenzyl salicylate, homomenthyl salicylate; derivatives of benzophenone, preferably 2-hydroxy-4-m ethoxybenzophenone, 2-hydroxy-4-m ethoxy-4′-methylbenzophenone, 2,2′-dihydroxy-4-methoxybenzophenone; esters of benzalmalonic acid, preferably di(2-ethylhexyl) 4-methoxybenzalmalonate, 2,4,6-trianilino-(p-carbo-2′-ethyl-1′-hexyloxy)-1,3,5-triazine. Advantageous water-soluble UVB filters include salts of 2-phenylbenzimidazole-5-sulphonic acid, such as its sodium, potassium or triethanolammonium salts, as well as the sulphonic acid itself; sulphonic acid derivatives of benzophenones, preferably 2-hydroxy-4-m ethoxybenzophenone-5-sulphonic acid and its salts; sulphonic acid derivatives of 3-benzylidene camphor, such as for example 4-(2-oxo-3-bornylidenemethyl)benzenesulphonic acid, 2-methyl-acid and their salts and also 1,4-di(2-oxo-10sulphato compounds, such as the corresponding sodium, potassium and its salts (the corresponding triethanolammonium salts), and benzene-1,4-di(2-oxo-3-bornylidenemethyl-10-sulphonic acid.

It can also be advantageous to employ UVA filters, such as are customarily contained in cosmetic preparations. These substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)propane-1,3-dione. The amounts used for the UVB combination can be used analogously.

A high content of treatment substances is usually advantageous in the beta-glucan composition or the preparation, comprising the beta-glucan composition, according to the present invention for the cosmetic treatment of the skin, hair, scalp or nails. In accordance with a preferred embodiment, the beta-glucan composition or the preparation, comprising the beta-glucan composition, according to the present invention contain one or more animal and/or vegetable treatment fats and oils, such as olive oil, sunflower oil, purified soybean oil, palm oil, sesame oil, rapeseed oil, almond oil, borage oil, evening primrose oil, coconut oil, shea butter, jojoba oil, sperm oil, beef tallow, neatsfoot oil and lard, and optionally other treatment constituents such as for example C8- to C30 fatty alcohols. The fatty alcohols used here can be saturated or unsaturated and straight-chain or branched, wherein examples include decanol, decenol, octanol, octenol, dodecanol, dodecenol, octadienol, decadienol, dodecadienol, oleyl alcohol, ricinoleyl alcohol, erucic alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, as well their guerbet alcohols; this list may be extended as desired to include other alcohols which structurally are chemically related. The fatty alcohols preferably originate from natural fatty acids and are usually prepared from the corresponding esters of the fatty acids by reduction. Fatty alcohol fractions formed by reduction from naturally occurring fats and fat oils can also be used, such as for example beef tallow, peanut oil, colza oil, cottonseed oil, soybean oil, sunflower oil, palm kernel oil, linseed oil, maize oil, castor oil, rapeseed oil, sesame oil, cocoa butter and cocoa fat. The treatment substances that can preferably be combined with the preparation according to the present invention can also include: phospholipids, for example soy lecithin, egg lecithin and cephalins; vaseline, paraffin and silicone oils, the latter including inter alia dialkyl- and alkylaryl-siloxanes such as dimethylpolysiloxane and methylphenylpolysiloxane, as well as their alkoxylated and quaternised derivatives.

Hydrolysed animal and/or vegetable proteins can also advantageously be added to the beta-glucan composition or the preparation, comprising the beta-glucan composition, according to the present invention. Advantageous examples in this regard include in particular elastin, collagen, keratin, lactoprotein, soy protein, oat protein, pea protein, almond protein and wheat protein fractions or corresponding hydrolysed proteins, as well as their condensation products with fatty acids, and also quaternised hydrolysed proteins, wherein the use of hydrolysed vegetable proteins is preferred.

If the beta-glucan composition or the preparation, comprising the beta-glucan composition, according to the present invention is a solution or lotion, then solvents which can be used include: water or aqueous solutions; fatty oils, fats, waxes and other natural and synthetic fatty bodies, preferably esters of fatty acids with alcohols having a low C number, such as isopropanol, propylene glycol or glycerol, or esters of fatty alcohols with alkanoic acids having a low C number or with fatty acids; alcohols, diols or polyols having a low C number, and their ethers, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products. Mixtures of the abovementioned solvents are in particular used. In the case of alcoholic solvents, water can be an additional constituent.

The beta-glucan composition or the preparation, comprising the beta-glucan composition, preferably a cosmetic or pharmaceutical preparation, according to the present invention can also contain vitamins and vitamin precursors, wherein any vitamins and vitamin precursors which are suitable or customary in cosmetic or pharmaceutical applications can be used. Particular mention may be made here of vitamins and vitamin precursors such as tocopherols, Vitamin A, nicotinic acid and nicotinamide, other B-complex vitamins, in particular biotin, and Vitamin C. Other examples within this group which are preferably used include pantothenyl alcohol and its derivatives, in particular its esters and ethers, as well as derivatives of pantothenyl alcohols obtained cationically, such as for example pantothenyl alcohol triacetate, pantothenyl alcohol monoethyl ether and its monoacetate and also cationic pantothenyl alcohol derivatives.

The beta-glucan composition or the preparation, comprising the beta-glucan composition, preferably a cosmetic or pharmaceutical preparation, according to the present invention can also contain active compounds having a skin-lightening action, wherein any skin-lightening active compounds that are suitable or customary in cosmetic or pharmaceutical applications can be used in accordance with the invention. Advantageous skin-lightening active compounds in this regard include kojic acid, hydroquinone, arbutin, ascorbic acid, magnesium ascorbyl phosphate, resorcinols, liquorice root extracts and their constituents glabridin or licochalcone A, or extracts from Rumex and Ramulus species, extracts from pine species (Pinus) or extracts from Vitis species which contain inter alia skin-lightening stilbene derivatives.

The beta-glucan composition or the preparation, comprising the beta-glucan composition, preferably a cosmetic or pharmaceutical preparation, according to the present invention can also contain mono-, di- and oligo-saccharides such as for example glucose, galactose, fructose, mannose and lactose.

The beta-glucan composition or the preparation, comprising the beta-glucan composition, preferably a cosmetic or pharmaceutical preparation, according to the present invention can also contain plant extracts, which are usually prepared by extraction of the complete plant, but which in individual cases are also prepared exclusively from the blossom and/or leaves, wood, bark or roots of the plant. Particularly advantageous extracts include aloe, Hamamelis, algae, oak bark, willow-herb, stinging nettles, dead nettles, hops, camomile, milfoil, arnica, calendula, burdock root, horse-tail, hawthorn, linden blossom, cucumber, almonds, pine needles, horse chestnut, sandalwood, juniper, coconut, mango, apricot, orange, lemon, lime, grapefruit, apple, green tea, grapefruit seed, wheat, oats, barley, sage, thyme, basil, rosemary, birch, mallow, bitter-crass, willow bark, restharrow, coltsfoot, althaea, ginseng and ginger root. Of these, particularly preferred extracts include aloe vera, camomile, algae, rosemary, calendula, ginseng, cucumber, sage, stinging nettles, linden blossom, arnica and Hamamelis. Mixtures of two or more plant extracts can also be employed. Extraction agents that can be used for preparing said plant extracts include water, alcohols and mixtures thereof. Preferred alcohols in this context are the lower alcohols such as ethanol and isopropanol, but also polyhydric alcohols such as ethylene glycol, propylene glycol and butylene glycol, specifically both as a sole extracting agent and in mixtures with water. Extracts, fractions and active substances from camomile, aloe vera, Commiphora species, Rubia species, willows, willow-herb, ginger, marigold, arnica, Glycyrrhiza species, Echinacea species, Rubus species and pure substances such as inter alia bisabolol, apigenin, apigenin-7-glucoside, gingerols such as [6]-gingerol, paradols such as [6]-paradol, boswellic acid, phytosterols, glycyrrhizine, glabridin or licochalcone A are particularly preferred. The plant extracts can be used in pure form or dilute form in accordance with the invention.

The beta-glucan composition or the preparation, comprising the beta-glucan composition, preferably a cosmetic or pharmaceutical preparation, according to the present invention may also include at least one further fragrance substance. The following specified fragrance substances can be used, either as individual substances or in mixtures with at least one, two, three or even more fragrance substances, in a large number of fragrance mixtures, selected from an extensive range of natural and synthetic substances.

Fragrance substances which are advantageously suitable for combining are the following examples of known odourant substances:
extracts of natural raw materials such as essential oils, concretes, absolutes, resins, resinoids, balsams, tinctures such as for example: ambergris tincture; amyris oil; angelica seed oil; angelica root oil; anise oil; valerian oil; basil oil; tree moss absolute; bay oil; artemisia oil; benzoin resin; bergamot oil; beeswax absolute; birch tar oil; bitter almond oil; savory oil; buchu leaf oil; cabreuva oil; cade oil; calamus oil; camphor oil; cananga oil; cardamom oil; cascarilla oil; cassia oil; cassie absolute; castoreum absolute cedar leaf oil; cedarwood oil; cistus oil; citronella oil; lemon oil; copaiba balsam; copaiba balsam oil; coriander oil; costus root oil; cumin oil; cypress oil; davana oil; dill weed oil; dill seed oil; eau de brouts absolute; oak moss absolute; elemi oil; tarragon oil; eucalyptus citriodora oil; eucalyptus oil; fennel oil; pine-needle oil; galbanum oil; galbanum resin; geranium oil; grapefruit oil; guaiac wood oil; gurjun balsam; gurjun balsam oil; helichrysum absolute; helichrysum oil; ginger oil; iris root absolute; iris root oil; jasmine absolute; calamus oil; blue camomile oil; Roman camomile oil; carrot seed oil; cascarilla oil; pine-needle oil; spearmint oil; caraway oil; labdanum oil; labdanum absolute; labdanum resin; lavandin absolute; lavandin oil; lavender absolute; lavender oil; lemongrass oil; lovage oil; distilled lime oil; pressed lime oil; linaloe oil; Litsea cubeba oil; bay leaf oil; mace oil; marjoram oil; mandarin oil; massoia bark oil; mimosa absolute; ambrette oil; musk tincture; muscatel sage oil; nutmeg oil; myrrh absolute; myrrh oil; myrtle oil; clove leaf oil; clove bud oil; neroli oil; olibanum absolute; olibanum oil; opopanax oil; orange blossom absolute; orange oil; origanum oil; palmarosa oil; patchouli oil; perilla oil; Peru balsam oil; parsley leaf oil; parsley seed oil; petitgrain oil; peppermint oil; pepper oil; pimento oil; pine oil; pennyroyal oil; rose absolute; rosewood oil; rose oil; rosemary oil; Dalmatian sage oil; Spanish sage oil; sandalwood oil; celery seed oil; spike lavender oil; star anise oil; styrax oil; tagetes oil; fir needle oil; tea tree oil; turpentine oil; thyme oil; Tolu balsam; tonka absolute; tuberose absolute; vanilla extract; violet leaf absolute; verbena oil; vetiver oil; juniper berry oil; cognac oil; wormwood oil; wintergreen oil; ylang ylang oil; hyssop oil; civet absolute; cinnamon leaf oil; cinnamon bark oil, and fractions thereof or constituents isolated therefrom;
individual fragrance substances from the group comprising hydrocarbons, such as for example 3-carene; alpha-pinene; beta-pinene; alpha-terpinene; gamma-terpinene; p-cymene; bisabolene; camphene; caryophyllene; cedrene; famesene; limonene; longifolene; myrcene; ocimene; valencene; (E,Z)-1,3,5-undecatriene; styrene; diphenylmethane;
aliphatic alcohols such as for example hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixtures of 3,4,5,6,6-pentamethyl-3,4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
aliphatic aldehydes and the acetals thereof such as for example hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanal diethylacetal; 1,1-dimethoxy-2,2,5-trim ethyl-4-hexene; citronellyloxyacetaldehyde; 1-(1-methoxypropoxy)-(E/Z)-3-hexene;
aliphatic ketones and the oximes thereof such as for example 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;
aliphatic sulphur-containing compounds such as for example 3-methylthio-hexanol; 3-methylthiohexyl acetate; 3-mercaptohexanol; 3-mercaptohexyl acetate; 3-mercaptohexyl butyrate; 3-acetylthiohexyl acetate; 1-menthen-8-thiol;
aliphatic nitriles such as for example 2-nonenoic acid nitrile; 2-undecenoic acid nitrile; 2-tridecenoic acid nitrile; 3,12-tridecadienoic acid nitrile; 3,7-dimethyl-2,6-octadienoic acid nitrile; 3,7-dimethyl-6-octenoic acid nitrile;
esters of aliphatic carboxylic acids such as for example (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl-isobutyrate; hexyl crotonate; ethyl isovalerate; ethyl-2-methyl pentanoate; ethyl hexanoate; allyl hexanoate; ethyl heptanoate; allyl heptanoate; ethyl octanoate; ethyl-(E,Z)-2,4-decadienoate; methyl-2-octinate; methyl-2-noninate; allyl-2-isoamyloxyacetate; methyl-3,7-dimethyl-2,6-octadienoate; 4-methyl-2-pentyl-crotonate;
acyclic terpene alcohols such as for example: citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2-butenoates thereof; acyclic terpene aldehydes and ketones such as for example geranial; neral; citronellal; 7-hydroxy-3,7-dimethyloctanal; 7-methoxy-3,7-dimethyloctanal; 2,6,10-trimethyl-9-undecenal; geranyl acetone; and the dimethyl and diethyl acetals of geranial, neral, 7-hydroxy-3,7-dimethyloctanal;
cyclic terpene alcohols such as for example: menthol; isopulegol; alpha-terpineol; terpinenol-4; menthan-8-ol; menthan-1-ol; menthan-7-ol; borneol; isoborneol; linalool oxide; nopol; cedrol; ambrinol; vetiverol; guaiol; and the formates, acetates, propionates, isobutyrates, butyrates, isovalerates, pentanoates, hexanoates, crotonates, tiglinates and 3-methyl-2-butenoates thereof;
cyclic terpene aldehydes and ketones such as for example menthone; isomenthone; 8-mercaptomenthan-3-one; carvone; camphor; fenchone; alpha-ionone; beta-ionone; alpha-n-methyl ionone; beta-n-methyl ionone; alpha-isomethyl ionone; beta-isomethyl ionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalen-8(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedarwood oil (methylcedryl ketone);
cyclic alcohols such as for example: 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
cycloaliphatic alcohols such as for example alpha-3,3-trimethylcyclohexylmethanol; 1-(4-isopropylcyclohexyl)ethanol; 2-methyl-4-(2,2,3-trim ethyl-3-cyclopent-1-yl)butanol; 2-methyl-4-(2,2,3-trimethyl-3-cyclopent-1-yl)-2-buten-1-ol; 2-ethyl-4-(2,2,3-trim ethyl-3-cyclopent-1-yl)-2-buten-1-ol; 3-methyl-5-(2,2,3-trimethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol; cyclic and cycloaliphatic ethers such as for example: cineole; cedryl methyl ether; cyclododecyl methyl ether; 1,1-dimethoxycyclododecane; (ethoxymethoxy)cyclododecane; alpha-cedrene epoxide; 3a,6,6,9a-tetramethyldodecahydronaphtho[2,1-b]furan; 3a-ethyl-6,6,9a-trimethyldodeca-hydronaphtho[2,1-b]furan; 1,5,9-trimethyl-13-oxabicyclo[10.1.0]trideca-4,8-diene; rose oxide; 2-(2,4-dimethyl-3-cyclohexen-1-yl)-5-methyl-5-(1-methylpropyl)-1,3-dioxane;
cyclic and macrocyclic ketones such as for example 4-tert-butylcyclohexanone; 2,2,5-trimethyl-5-pentylcyclopentanone; 2-heptylcyclopentanone; 2-pentylcyclo-pentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 8-cyclohexadecen-1-one; 9-cycloheptadecen-1-one; cyclopentadecanone; cyclohexadecanone;
cycloaliphatic aldehydes such as for example 2,4-dimethyl-3-cyclohexene carbaldehyde; 2-methyl-4-(2,2,6-trimethyl-cyclohexen-1-yl)-2-butenal; 4-(4-hydroxy-4-m ethylpentyl)-3-cyclohexene carbaldehyde; 4-(4-methyl-3-penten-1-yl)-3-cyclohexene carbaldehyde;
cycloaliphatic ketones such as for example 1-(3,3-dimethyl-cyclohexyl)-4-penten-1-one; 2,2-dimethyl-1-(2,4-dimethyl-3-cyclohexene-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl-2,6,10-trimethyl-2,5,9-cyclododecatrienyl ketone; tert-butyl-(2,4-dimethyl-3-cyclohexen-1-yl) ketone;
esters of cyclic alcohols such as for example 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- or 6-indenyl acetate; 4,7-methano-3a,4,5,6,7,7a-hexahydro-5- or 6-indenyl propionate; 4,7-methano-3a,4,5,6,7,7a-hexahydro-5- or 6-indenyl isobutyrate; 4,7-methanooctahydro-5- or 6-indenyl acetate;
esters of cycloaliphatic alcohols such as for example 1-cyclohexylethyl crotonate; esters of cycloaliphatic carboxylic acids such as for example allyl-3-cyclohexyl propionate; allylcyclohexyl oxyacetate; cis- and trans-methyl dihydrojasmonate; cis- and trans-methyl jasmonate; methyl-2-hexyl-3-oxocyclopentane carboxylate; ethyl-2-ethyl-6,6-dimethyl-2-cyclohexene carboxylate; ethyl-2,3,6,6-tetramethyl-2-cyclohexene carboxylate; ethyl-2-methyl-1,3-dioxolane 2-acetate;
araliphatic alcohols such as for example benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
esters of araliphatic alcohols and aliphatic carboxylic acids such as for example benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha,alpha-dimethylphenylethyl acetate; alpha,alpha-dimethylphenyl-ethyl butyrate; cinnamyl acetate; 2-phenoxyethyl isobutyrate; 4-methoxybenzyl acetate; araliphatic ethers such as for example: 2-phenyl ethyl methyl ether; 2-phenyl ethyl isoamyl ether; 2-phenyl ethyl 1-ethoxyethyl ether; phenylacetaldehyde dimethylacetal; phenylacetaldehyde diethylacetal; hydratropaldehyde dimethylacetal; phenylacetaldehyde glycerol acetal; 2,4,6-trimethyl-4-phenyl-1,3-dioxane; 4,4a,5,9b-tetrahydroindeno[1,2-d]-m-dioxin; 4,4a,5,9b-tetrahydro-2,4-dimethylindeno[1,2-d]-m-dioxin;
aromatic and araliphatic aldehydes such as for example: benzaldehyde; phenylacetaldehyde; 3-phenylpropanal; hydratropaldehyde; 4-methylbenz-aldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 2-methyl-3-(4-isobutylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-m ethoxybenzaldehyde; 4-hydroxy-3-m ethoxybenzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;
aromatic and araliphatic ketones such as for example: acetophenone; 4-methyl-acetophenone; 4-methoxyacetophenone; 4-tert-butyl-2,6-dimethylacetophenone; 4-phenyl-2-butanone; 4-(4-hydroxyphenyl)-2-butanone; 1-(2-naphthalenyl)ethanone; 2-benzofuranylethanone; (3-methyl-2-benzofuranyl)ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone;
aromatic and araliphatic carboxylic acids and the esters thereof such as for example: benzoic acid; phenylacetic acid; methyl benzoate; ethyl benzoate; hexyl benzoate; benzyl benzoate; methylphenyl acetate; ethylphenyl acetate; geranylphenyl acetate; phenylethylphenyl acetate; methyl cinnamate; ethyl cinnamate; benzyl cinnamate; phenylethyl cinnamate; cinnamyl cinnamate; allyl phenoxy acetate; methyl salicylate; isoamyl salicylate; hexyl salicylate; cyclohexyl salicylate; cis-3-hexenyl salicylate; benzyl salicylate; phenylethyl salicylate; methyl-2,4-dihydroxy-3,6-dimethylbenzoate; ethyl-3-phenyl glycidate; ethyl-3-methyl-3-phenyl glycidate;
nitrogenous aromatic compounds such as for example: 2,4,6-trinitro-1,3-dimethyl-5-tert-butylbenzene; 3,5-dinitro-2,6-dimethyl-4-tert-butyl aceto-phenone; cinnamonitrile; 3-methyl-5-phenyl-2-pentenoic acid nitrile; 3-methyl-5-phenylpentanoic acid nitrile; methyl anthranilate; methyl-N-methyl anthranilate; Schiff bases of methyl anthranilate with 7-hydroxy-3,7-dimethyloctanal, 2-methyl-3-(4-tert-butylphenyl)propanal or 2,4-dimethyl-3-cyclohexene carbaldehyde 6-isopropyl quinoline; 6-isobutyl quinoline; 6-sec-butyl quinoline; 2-(3-phenylpropyl)pyridine; indole; skatole; 2-methoxy-3-isopropylpyrazine; 2-isobutyl-3-methoxypyrazine;
phenols, phenyl ethers and phenyl esters such as for example: estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenyl acetate;
heterocyclic compounds such as for example: 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
lactones such as for example: 1,4-octanolide; 3-methyl-1,4-octanolide; 1,4-nonanolide; 1,4-decanolide; 8-decen-1,4-olide; 1,4-undecanolide; 1,4-dodecan-olide; 1,5-decanolide; 1,5-dodecanolide; 4-methyl-1,4-decanolide; 1,15-penta-decanolide; cis- and trans-11-pentadecen-1,15-olide; cis- and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin;
and mixtures of the above substances.

The beta-glucan composition or the preparation, that contain the beta-glucan composition, preferably a cosmetic or pharmaceutical preparation, according to the present invention can also contain anionic, cationic, non-ionic and/or amphoteric surfactants, especially if crystalline or microcrystalline solids, for example inorganic micropigments, are to be incorporated into the preparations according to the present invention. Surfactants are amphiphilic substances that are able to dissolve organic, non-polar substances in water. Surfactants are generally classified according to the nature and charge of the hydrophilic part of the molecule. Four groups can be differentiated here: anionic surfactants, cationic surfactants, amphoteric surfactants and non-ionic surfactants.

Anionic surfactants usually contain carboxylate, sulphate or sulphonate groups as functional groups. In aqueous solution, they form negatively charged organic ions in the acid or neutral medium. Cationic surfactants are characterised almost exclusively by the presence of a quaternary ammonium group. In aqueous solution, they form positively charged organic ions in the acid or neutral medium. Amphoteric surfactants contain both anionic and cationic groups and accordingly behave like anionic or cationic surfactants in aqueous solution, depending on the pH value. They have a positive charge in a strongly acid medium and a negative charge in an alkaline medium. In the neutral pH range, by contrast, they are zwitterionic. Polyether chains are typical of non-ionic surfactants. Non-ionic surfactants do not form ions in an aqueous medium.

Anionic surfactants that can advantageously be used include: acyl amino acids (and their salts), such as: acyl glutamates, for example sodium acyl glutamate, di-TEA-palmitoyl aspartate and sodium caprylic/capric glutamate; acyl peptides, for example palmitoyl-hydrolysed lactoprotein, sodium cocoyl-hydrolysed soy protein and sodium/potassium cocoyl-hydrolysed collagen; sarcosinates, for example myristoyl sarcosinate, TEA-lauroyl sarcosinate, sodium lauroyl sarcosinate and sodium cocoyl sarcosinate; taurates, for example sodium lauroyl taurate and sodium methyl cocoyl taurate; acyl lactylates, for example lauroyl lactylate and caproyl lactylate; alaninates; carboxylic acids and derivatives, such as for example lauric acid, aluminium stearate, magnesium alkanolate and zinc undecylenate; ester carboxylic acids, for example calcium stearoyl lactylate, laureth-6 citrate and sodium PEG-4 lauramide carboxylate; ether carboxylic acids, for example sodium laureth-13 carboxylate and sodium PEG-6 cocamide carboxylate; phosphoric acid esters and salts, such as for example DEA-oleth-10 phosphate and dilaureth-4 phosphate; sulphonic acids and salts, such as acyl isethionates, for example sodium/ammonium cocoyl isethionate; alkyl aryl sulphonates; alkyl sulphonates, for example sodium cocomonoglyceride sulphonate, sodium C12-14 olefin sulphonate, sodium lauryl sulphoacetate and magnesium PEG-3 cocamide sulphate; sulphosuccinates, for example dioctyl sodium sulphosuccinate, disodium laureth sulphosuccinate, disodium lauryl sulphosuccinate and disodium undecylenamido MEA-sulphosuccinate; and sulphuric acid esters, such as alkyl ether sulphate, for example sodium, ammonium, magnesium, MIPA, TIPA laureth sulphate, sodium myreth sulphate and sodium C12-13 pareth sulphate, and alkyl sulphates, for example sodium, ammonium and TEA lauryl sulphate.

Cationic surfactants that can advantageously be used include: alkyl amines, alkyl imidazoles, ethoxylated amines and quaternary surfactants.

Quaternary surfactants contain at least one N atom that is covalently bonded to four alkyl or aryl groups. This leads to a positive charge, irrespective of the pH value. Alkyl betaine, alkyl amidopropyl betaine and alkyl amidopropyl hydroxysulphaine are advantageous. The cationic surfactants used can also preferably be chosen from the group of quaternary ammonium compounds, in particular benzyl trialkyl ammonium chlorides or bromides, such as for example benzyl dimethylstearyl ammonium chloride, as well as alkyl trialkyl ammonium salts, for example cetyl trimethyl ammonium chloride or bromide, alkyl dimethyl hydroxyethyl ammonium chlorides or bromides, dialkyl dimethyl ammonium chlorides or bromides, alkyl amide ethyl trimethyl ammonium ether sulphates, alkyl pyridinium salts, for example lauryl or cetyl pyridinium chloride, imidazoline derivatives and compounds of a cationic nature, such as amine oxides, for example alkyl dimethyl amine oxides or alkyl aminoethyl dimethyl amine oxides. Cetyl trimethyl ammonium salts can particularly advantageously be used.

Amphoteric surfactants that can advantageously be used include: acyl/dialkyl ethylene diamine, for example sodium acyl amphoacetate, disodium acyl amphodipropionate, disodium alkyl amphodiacetate, sodium acyl amphohydroxypropyl sulphonate, disodium acyl amphodiacetate and sodium acyl amphopropionate; N-alkyl amino acids, for example aminopropyl alkyl glutamide, alkyl aminopropionic acid, sodium alkyl imidodipropionate and lauroamphocarboxyglycinate.

Non-ionic surfactants that can advantageously be used include: alcohols; alkanolamides, such as cocamides MEA/DEA/MIPA, amine oxides, such as cocoamidopropylamine oxide; esters formed by esterification of carboxylic acids with ethylene oxide, glycerol, sorbitan or other alcohols; ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated/propoxylated lanolin, ethoxylated/propoxylated polysiloxanes, propoxylated POE ethers and alkyl polyglycosides, such as lauryl glucoside, decyl glycoside and cocoglycoside; sucrose esters and ethers; polyglycerol esters, diglycerol esters, monoglycerol esters; methyl glucose esters, ester of hydroxy acids.

The use of a combination of anionic and/or amphoteric surfactants with one or more non-ionic surfactants is also advantageous. The surface-active substance can be present in a concentration of between 1 and 98% (m/m) in the preparations containing histamine-release inhibitors in accordance with the invention, based on the dry weight of the preparations.

The beta-glucan composition or the preparation, comprising the beta-glucan composition, preferably a cosmetic or pharmaceutical preparation, according to the present invention can also contain active compounds for preservative purposes, wherein any preservatives may be used which are suitable or customary in cosmetic or pharmaceutical, in particular dermatological, applications and which are advantageously selected from the group consisting of preservatives such as inter alia benzoic acid, its esters and salts; propionic acid and its salts; salicylic acid and its salts; 2,4-hexanoic acid (sorbic acid) and its salts; formaldehyde and paraformaldehyde; 2-hydroxybiphenyl ether and its salts; 2-zincsulphidopyridine N-oxide; inorganic sulphites and bisulphites; sodium iodate; chlorobutanol; 4-hydroxybenzoic acid and its salts and esters; dehydroacetic acid; formic acid; 1,6-bis(4-amidino-2-bromophenoxy)-n-hexane and its salts; the sodium salt of ethylmercury-(II)-thiosalicylic acid; phenylmercury and its salts; 10-undecylenic acid and its salts; 5-amino-1,3-bis(2-ethylhexyl)-5-methylhexahydropyrimidine; 5-bromo-5-nitro-1,3-dioxane; 2-bromo-2-nitro-1,3-propanediol; 2,4-dichlorobenzyl alcohol; N-(4-chlorophenyl)-N′-(3,4-dichlorophenyl)urea; 4-chloro-m-cresol; 2,4,4′-trichloro-2′-hydroxy-diphenyl ether; 4-chloro-3,5-dimethylphenol; 1,1′-methylene-bis(3-(1-hydroxymethyl-2,4-dioximidazolidin-5-yl)urea); poly(hexamethylene biguanide) hydrochloride; 2-phenoxyethanol; hexamethylenetetramine; 1-(3-chloroallyl)-3,5,7-triaza-1-azoniaadamantane chloride; 1-(4-chloro-phenoxy)-1(1H-imidazol-1-yl)-3,3-dimethyl-2-butanone; 1,3-bis(hydroxymethyl)-5,5-dimethyl-2,4-imidazolidinedione; benzyl alcohol; Octopirox®; 1,2-dibromo-2,4-dicyanobutane; 2,2′-methylene-bis(6-bromo-4-chloro-phenol); bromochlorophene; mixture of and 2-methyl-3(2H)isothiazolinone with magnesium chloride and magnesium nitrate; 2-benzyl-4-chlorophenol; 2-chloroacetamide; chlorhexidine; chlorhexidine acetate; chlorhexidine gluconate; chlorhexidine hydrochloride; 1-phenoxy-propan-2-ol; N-alkyl(C12-C22)trimethylammonium bromide and chloride; 4,4-dimethyl-1,3-oxazolidine; N-hydroxymethyl-N-(1,3-di(hydroxymethyl)-2,5-dioxoimidazolidin-4-yl)-N′-hydroxymethylurea; 1,6-bis(4-amidinophenoxy)-n-hexane and its salts; glutaraldehyde 5-ethyl-1-aza-3,7-dioxabicyclo(3.3.0)octane; 3-(4-chlorophenoxy)-1,2-propanediol; hyamine; alkyl(C8-C18)dimethylbenzylammonium chloride; alkyl(C8-C18)dimethylbenzylammonium bromide; alkyl(C8-C18)dimethylbenzylammonium saccharinate; benzylhemiformal; 3-iodo-2-propynyl butylcarbamate; sodium ((hydroxymethyl)amino)acetate; and pentylene glycol.

The beta-glucan composition or the preparation, comprising the beta-glucan composition, preferably a cosmetic or pharmaceutical preparation, according to the present invention can also contain antioxidants, wherein any antioxidants suitable or customary in cosmetic or pharmceutical applications can be used. Advantageously, the antioxidants are selected from the group consisting of amino acids (for example glycine, histidine, tyrosine, tryptophan) and their derivatives, imidazoles (for example urocanic acid) and their derivatives, peptides such as D,L-carnosine, D-carnosine, L-carnosine and their derivatives (for example anserine), carotenoids, carotenes (for example α-carotene, β-carotene, lycopene) and their derivatives, lipoic acid and its derivatives (for example dihydrolipoic acid), aurothioglucose, propylthiouracil and other thiols (for example thioredoxin, glutathione, cysteine, cystine, cystamine and their glycosyl, N-acetyl, methyl, ethyl, propyl, amyl, butyl and lauryl, palmitoyl, oleyl, γ-linoleyl, cholesteryl and glyceryl esters) and their salts, dilauryl thiodipropionate, distearyl thiodipropionate, thiodipropionic acid and their derivatives (esters, ethers, peptides, lipids, nucleotides, nucleosides and salts) as well as sulphoximine compounds (for example buthionine sulphoximines, homocysteine sulphoximines, buthionine sulphones, penta-, hexa-, hepta-thionine sulphoximine) in very low tolerated doses, and also (metal) chelating agents, for example α-hydroxy fatty acids, palmitic acid, phytic acid, lactoferrin, α-hydroxy acids (for example citric acid, lactic acid, malic acid), humic acid, bile acid, bile extracts, bilirubin, biliverdin, EDTA, EGTA and their derivatives, unsaturated fatty acids and their derivatives (for example γ-linolenic acid, linoleic acid, oleic acid), folic acid and its derivatives, ubiquinone and ubiquinol and their derivatives, Vitamin C and its derivatives (for example ascorbyl palmitate, magnesium ascorbyl phosphate, ascorbyl acetate), tocopherols and their derivatives (for example Vitamin E acetate), Vitamin A and its derivatives (for example Vitamin A palmitate) and also coniferyl benzoate of benzoin resin, rutinic acid and its derivatives, ferrulic acid and its derivatives, butylhydroxytoluene, butylhydroxyanisole, nordihydroguaiacic acid, nordihydroguaiaretic acid, trihydroxybutyrophenone, uric acid and its derivatives, mannose and its derivatives, zinc and its derivatives (for example ZnO, ZnSO₄), selenium and its derivatives (such as selenium methionine), stilbenes and their derivatives (such as stilbene oxide, trans-stilbene oxide), as well as the derivatives (such as salts, esters, ethers, sugars, nucleotides, nucleosides, peptides and lipids) of said active compounds such as are suitable in accordance with the invention.

The beta-glucan composition or food, food supplement, cosmetic, pharmaceutical or veterinary preparation, comprising the beta-glucan composition, according to the present invention, preferably a cosmetic or pharmaceutical preparation, may also contain one or more emulsifiers commonly used in the art for preparing a food, food supplement cosmetic, pharmaceutical or veterinary preparation.

The beta-glucan composition or the preparation, comprising the beta-glucan composition, preferably a cosmetic or pharmaceutical preparation, according to the present invention may also include a cosmetically or pharmaceutically acceptable carrier, such as (without being limited to) one of the following which are commonly used in the art: lactose, dextrose, sucrose, sorbitol, mannitol, starch, acacia rubber, calcium phosphate, alginate, gelatine, calcium silicate, microcrystalline cellulose, polyvinyl pyrrolidone, cellulose, water, syrup, methyl cellulose, methyl hydroxybenzoate, propyl hydroxybenzoate, talc, magnesium stearate, mineral oil and the like. The cosmetic or pharmaceutical, in particular dermatological, preparation may also include lubricants, wetting agents, sweeteners, flavouring agents, emulsifiers, suspensions, preserving agents and the like, in addition to the above components. Suitable pharmaceutically acceptable carriers and preparations are described in detail in Remington's Pharmaceutical Sciences (19^{th} edition, 1995).

The beta-glucan composition or the preparation, comprising the beta-glucan composition, preferably a cosmetic or pharmaceutical preparation, according to the present invention can be easily formulated as conventional cosmetic or pharmaceutical preparations for personal care, preferably for skin, hair, scalp and nail care.

In a preferred variant, the present invention relates thus to cosmetic or pharmaceutical, preferably dermatological, preparations, in particular to preparations for personal care, preferably for skin, hair, scalp and nail care.

In a further variant, the present invention relates to preparations for pet care.

Within the context of the present invention, the beta-glucan composition or the preparation, comprising the beta-glucan composition, preferably a cosmetic or pharmaceutical preparation, according to the present invention can be provided either in liquid or solid form. Preferably, the preparations according to the present invention can take various forms such as are for example customarily employed for this type of preparations and suitable for topical application, for example as lotions, aqueous or aqueous-alcoholic gels, vesicle dispersions, or as simple or complex emulsions (O/W, W/O, O/W/O or W/O/W), liquids, semi-liquids or solids, such as milks, creams, gels, cream-gels, pastes or sticks, and can optionally be packaged as an aerosol and take the form of mousses or sprays. These preparations are prepared according to usual methods.

In a preferred variant, the cosmetic or pharmaceutical, in particular dermatological, preparations according to the present invention are selected from the group consisting of solid soap, liquid soap, cleansing products, cleansing gel, bath and shower additives, hair, skin and body shampoo, hair conditioner, shaving products, antidandruff shampoo and micellar water.

For preparing emulsions, the oil phase can advantageously be chosen from the following group of substances: mineral oils, mineral waxes; fatty oils, fats, waxes and other natural and synthetic fatty bodies, preferably esters of fatty acids with alcohols having a low C number, for example with isopropanol, propylene glycol or glycerol, or esters of fatty alcohols with alkanoic acids having a low C number or with fatty acids; alkyl benzoates; silicone oils such as dimethyl polysiloxanes, diethyl polysiloxanes, diphenyl polysiloxanes and mixed forms thereof. Advantageously, esters of saturated and/or unsaturated, branched and/or straight-chain alkane carboxylic acids having a chain length of 3 to 30 C atoms and saturated and/or unsaturated, branched and/or straight-chain alcohols having a chain length of 3 to 30 C atoms, from the group of esters of aromatic carboxylic acids and saturated and/or unsaturated, branched and/or straight-chain alcohols having a chain length of 3 to 30 C atoms can be used. Preferred ester oils include isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of such esters, for example jojoba oil.

The oil phase can also advantageously be chosen from the group comprising branched and straight-chain hydrocarbons and waxes, silicone oils, dialkyl ethers, the group comprising saturated or unsaturated, branched or straight-chain alcohols, and fatty acid triglycerides, specifically triglycerol esters of saturated and/or unsaturated, branched and/or straight-chain alkane carboxylic acids having a chain length of 8 to 24, in particular 12 to 18 C atoms. The fatty acid triglycerides can for example advantageously be chosen from the group comprising synthetic, semi-synthetic and natural oils, for example olive oil, sunflower oil, soybean oil, peanut oil, rapeseed oil, almond oil, palm oil, coconut oil, palm kernel oil and the like. Arbitrary admixtures of such oil and wax components can also advantageously be used. In some cases, it is also advantageous to use waxes, for example cetyl palmitate, as the sole lipid component of the oil phase; the oil phase is advantageously chosen from the group consisting of 2-ethylhexyl isostearate, octyldodecanol, isotridecyl isononanoate, isoeicosane, 2-ethylhexyl cocoate, C12-15 alkyl benzoate, caprylic-capric acid triglyceride and dicaprylyl ether. Mixtures of C12-15 alkyl benzoate and 2-ethylhexyl isostearate, mixtures of C12-15 alkyl benzoate and isotridecyl isononanoate and mixtures of C12-15 alkyl benzoate, 2-ethylhexyl isostearate and isotridecyl isononanoate are particularly advantageous. The hydrocarbons paraffin oil, squalane and squalene can also advantageously be used. The oil phase can advantageously also contain or consist entirely of cyclic or linear silicone oils, although an additional content of other oil phase components in addition to the silicone oil or oils is preferably used. Cyclomethicone (for example, decamethylcyclopentasiloxane) can advantageously be used as the silicone oil. However, other silicone oils can also advantageously be used, such as for example undecamethylcyclotrisiloxane, polydimethylsiloxane and poly(methylphenylsiloxane). Mixtures of cyclomethicone and isotridecyl isononanoate and of cyclomethicone and 2-ethylhexyl isostearate are also particularly advantageous.

The aqueous phase of preparations that contain the composition or oat extract according to the present invention and are provided in the form of an emulsion can include: alcohols, diols or polyols having a low C number, and their ethers, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, as well as alcohols having a low C number, such as ethanol, isopropanol, 1,2-propanediol, glycerol and in particular one or more thickeners, which can advantageously be chosen from the group comprising silicon dioxide, aluminium silicates, polysaccharides and their derivatives, such as hyaluronic acid, xanthan gum, hydroxypropyl methyl cellulose, and particularly advantageously from the group comprising polyacrylates, preferably a polyacrylate from the group comprising so-called carbopols, such as type 980, 981, 1382, 2984, 5984 carbopols, each on their own or in combinations.

The present invention shall now be described in detail with reference to the following examples, which are merely illustrative of the present invention, such that the content of the present invention is not limited by or to the following examples.

### Examples

Examples of the present invention are described below. The invention should not however be construed as being limited to the examples detailed.

In the following examples, the impact of different parameters on stability and gelation, of a beta-glucan solution is tested. The stability of the cereal beta-glucan comprising liquid composition, in particular gelation, is determined by freeze-thaw cycles. The test sample is subjected to a predetermined temperature-time profile simulating a freeze-thaw cycle by means of a temperature-equalization bath. The sample of the beta-glucan comprising liquid composition is placed in the −20° C. temperature-equalization bath and left in the bath for 12 h until frozen solid. The sample is next allowed to return to room temperature for 12 h, etc. Said freeze-thaw cycles are performed as long as gelation of the sample occurs. The higher the number of freeze-thaw cycles is, the more stable is the sample and gelation is retarded.

### Example 1: Influence of the pH Value on Gelation

The optimum pH range is tested with a well homogeneous aqueous solution of SymGlucan®, which is a 1% solution of oat beta-glucan in water, stabilized with glycerin, 1,2-hexanediol and caprylyl glycol. Solutions of sodium hydroxide or citric acid were added directly to the SymGlucan® solution under mixing and different pH values were adjusted. Thereafter, the samples were subjected to freeze-thaw cycles as described in detail above. The results are shown in Table 1.

TABLE 1
pH assays

| Trials | H | I | J |
|---|---|---|---|
| SymGlucan ®, batch No. 2, homogeneous, not | 3.10 | 5.10 | 9.10 |
| gelled, initial pH 4.71 | | | |
| Number of freeze/thaw cycles | >8 | >8 | 7 |

The above tests show, that gelation occurs at extreme pH values, especially under basic conditions. The sample J at a pH value of 9.10 gelled after 7 freeze/thaw cycles. After 13 cycles, samples H and I were compared: sample H was more aggregated than sample I. Conclusion: the optimal pH to formulate a beta-glucan composition is a pH value range from 4.5 to 6.0.

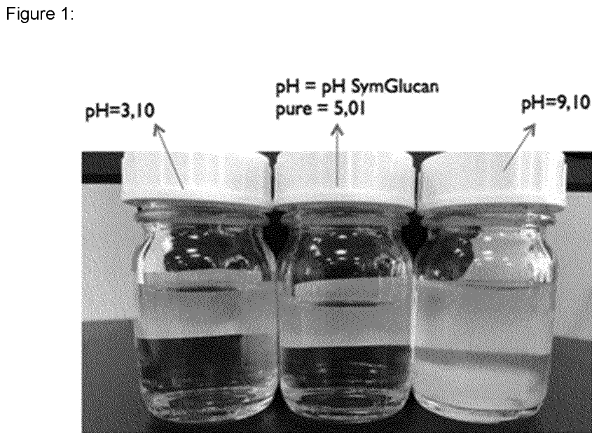

Additionally, as can be seen from FIG. 1, samples H and I (from left to the right), which were not subjected to freeze/thaw cycles, but stored at room temperature, did not gel even after 2 months. Sample J with a pH value of 9.10 gelled after 2 months storage.

### Example 2: Influence of Salts on Gelation

Different salts at different concentrations were tested with an aqueous SymGlucan® solution, as defined above. The blends were mixed with a magnetic stirrer for 15 min. The final pH value was adjusted to 4.5 to 5.0. Again, the samples were subjected to freeze/thaw cycles as described in detail above. The results are shown in Table 2.

TABLE 2
salts assays

| Trials | K | L | M | N | O | P | Q |
|---|---|---|---|---|---|---|---|
| SymGlucan ®, batch No. 1, | qs 100% | | | | | | |
| homogeneous, not gelled | | | | | | | |
| Biotive ®, L-Arginine (wt %) | | | | | | 1 | 0.5 |
| NaCl (wt %) | | 0.5 | 1 | 2 | 4 | | 0.5 |
| Number of freeze/thaw cycles | 6 | 7 | 9-10 | >11 | >11 | 6 | 6 |

The above tests show the impact of NaCl at different concentrations and L-Arginine at different concentrations on gelation delay. The sample K was utilized as reference control. The results indicate that the reference control solution gelled after 6 freeze/thaw cycles. Higher concentrations of NaCl (samples M, N and O) show the best results. At a concentration of NaCl of 1% by weight, improvement in gelation retard is noticeable. The samples with a NaCl concentration of 2 or 4% by weight underwent more than 11 freeze/thaw cycles without deterioration of product quality. Thus, the best performance can be found for aqueous SymGlucan® solutions which are stabilized with 2 or 4% by weight NaCl. However, no significant difference between 2% by weight and 4% by weight is observed. L-Arginine has not positive impact on gelation.

### Example 3: Influence of NaCl on Stability and Gelation

The influence of NaCl on stability and gelation was tested with an aqueous SymLift® solution, i.e. an aqueous solution comprising a barley extract.

After preparation of both batches, as described below, both batch samples were subjected to freeze/thaw cycles under the conditions as described in detail above. The results are shown in Table 3.

TABLE 3

| | SymLift ® pure | SymLift ® + 2% by |
|---|---|---|
| | (batch 1) | weight salt (batch 2) |
| Freeze/thaw cycle 1 | 0 | 0 |
| Freeze/thaw cycle 2 | 0 | 0 |
| Freeze/thaw cycle 3 | 0 | 0 |
| Freeze/thaw cycle 4 | 1 | 0 |
| Freeze/thaw cycle 5 | 2 | 1 |
| Freeze/thaw cycle 6 | 2 | 1 |
| Freeze/thaw cycle 7 | 3 | 1 |
| Freeze/thaw cycle 8 | 3 | 1 |
| Freeze/thaw cycle 9 | 3 | 1 |
| Freeze/thaw cycle 10 | 3 | 1 |
| Freeze/thaw cycle 11 | 3 | 2 |
| Freeze/thaw cycle 12 | 3 | 2.5 |
| Freeze/thaw cycle 13 | 3 | 2.5 |
| Freeze/thaw cycle 14 | 3 | 2.5 |

The above tests show the impact of NaCl on gelation delay of a SymLift® pure solution according to the present invention. The addition of NaCl to the liquid β-glucan comprising composition leads to a significant improvement of gelation delay. The solution comprising the NaCl withstands 11 freeze/thaw cycles compared to the same pure solution without salt which underwent 5 cycles until gelation only.

### Example 4: Influence of Salts on Gelation

In order to see the influence of electrostatic effects and steric hindrance, different salts were tested, especially chlorides. The tests were performed with a pure beta-glucan solution. The results are shown in Table 4.

TABLE 4
salts assays

| Trials | 2 | 13 | 14 | 15 | 16 | 17 | 18 | 20 | 22 |
|---|---|---|---|---|---|---|---|---|---|
| Water | | | | | | | | | |
| Hydrolite ®5 green | 10 | 6 | 6 | 6 | 6 | 6 | 6 | 6 | 6 |
| (pentylene glycol) | | | | | | | | | |
| Citric acid | 0.33 | qs | qs | | qs | qs | qs | qs | |
| Beta-glucan, unpreserved, | 89.7 | 94 | 92 | 92 | 92 | 93.6 | 91 | 93.6 | 91.3 |
| w/3% glycerine | | | | | | | | | |
| Glycerin | | | | | | | 3 | | |
| Sodium benzoate | | | | | | 0.4 | | | |
| NaCl | | | 2 | | | | | | |
| CaCl₂ dihydrate | | | | 2 | | | | | |
| KCl | | | | | 2 | | | | |
| NaOH (10%) | | | | qs | | | | | 0.69 |
| Dehydroacetate sodium | | | | | | | | 0.4 | |
| Levulinic acid | | | | | | | | | 0.4 |
| pH value | 4.78 | 4.9 | 4.87 | 4.87 | 4.9 | 4.9 | 4.93 | 4.99 | 4.47 |
| Beta-glucan content | 1.09 | 1.1 | 1.08 | 1.08 | 1.08 | 1.1 | 1.07 | 1.09 | 1.06 |
| Number of freeze/thaw cycles | 3 | 15 | >23 | 21 | >23 | 17 | 13 | | |

Note: the different assays started from different beta-glucan batches. Observations were realized by different technologists. Thus, slight variations are noticed. Basic classification is determined: 0=no gelation; 1=beginning of gel formation; 2=gel; 3=gel and salting out (gel contraction). When criterion 2 appears, the number of freeze/thaw cycles is determined.

Based on the number of freeze/thaw cycles needed to gel the samples, the following can concluded:
Amount of beta-glucan: a higher concentration of beta glucan accelerates the gelation. Larger amount of hygroscopic compounds (e.g. glycerin, Hydrolite® 5 green (pentylene glycol)) accelerates the gelation (sample 2 versus sample 13).
NaCl, CaCl₂) dihydrate and KCl at 2% by weight show distinguished performance (samples 14, 15 and 16).
No big difference is observed for different cations such as Na⁺, K⁺ and Ca²⁺ with regard to gelation time (samples 14, 15 and 16).
Dehydroacetate sodium has a negative impact due to recrystallization (sample 20).
Sodium benzoate improves the stability (sample 17 versus sample 13).
NaCl at 2% by weight in combination with Hydrolite® 5 green definitely shows superior performance (sample 14 versus sample 13).

1. A liquid composition comprising:
at least one cereal beta-glucan or a cereal extract comprising at least one cereal beta-glucan; and
at least one added inorganic and/or organic salt or a mixture thereof.

2. The composition according to claim 1, wherein the cereal beta-glucan is an oat beta-glucan.

3. The composition according to claim 1, wherein the total beta-glucan content is from 0.1 to 10.0% by weight based on the total weight of the composition.

4. The composition according to claim 1, wherein the inorganic salt consists of a monovalent or divalent metal and an anion of an inorganic acid or is a mixture of two or more of said inorganic salts; and/or the organic salt consists of a monovalent or divalent metal and an anion of an organic acid or is a mixture of two or more of said organic salts.

5. The composition according to claim 1, to wherein the added amount of salt in the composition is from 0.1 to 10% by weight, based on the total weight of the composition.

6. The composition according to claim 1, wherein the composition is a solution
wherein the maximum viscosity of the solution is from 100 to 50000 mPa·s, in particular 500 to 8000 to mPa·s, and/or
wherein the pH value of the solution is from 3.0 to 7.0, in particular 4.0 to 6.0.

7. The composition according to claim 1, optionally further comprising a multifunctional compound selected from the group consisting of Na benzoate, Na hyaluronate, polyols respective glycols, such as 1,2-propanediol (propylene glycol), 1,3-propanediol, 1,2-butanediol, 1,3-butanediol (butylene glycol), 1,2-pentanediol (pentylene glycol: Hydrolite® 5; green version or any grade), 1,2-hexanediol (Hydrolite® 6), 1,2-octanediol (caprylyl glycol; Hydrolite® 8), 1,2-decanediol (decylene glycol), or glycerol, phenoxyethanol, ethylhexylglycerin, glyceryl caprylate, hydroxyacetophenone, methylbenzyl alcohol, o-cymen-5-ol, benzyl alcohol, tropolone, and mixtures of two or more of said multifunctional compounds.

8. A method for producing a liquid cereal beta-glucan composition having retarded gelation, wherein said method comprises:
(a) providing a solution comprising at least one cereal beta-glucan or a cereal extract comprising at least one cereal beta-glucan;
(b) adding to the solution of step (a) at least one inorganic or organic salt or a mixture thereof to obtain a mixture;
(c) optionally adjusting the pH value of the mixture to a pH value in a range from 3.0 to 7.0; and
(d) optionally heating said mixture to a temperature in a range from 20° C. to 100° C.

9. A method for transforming a gelled cereal beta-glucan comprising composition, wherein said method comprises:
(i) providing a gelled composition comprising at least one cereal beta-glucan or a cereal extract comprising at least one cereal beta-glucan;
(ii) heating said gelled composition to a temperature in a range from 20° C. to 100° C. to obtain a solution;
(iii) adding to the solution of step (ii) at least one inorganic and/or organic salt or mixtures thereof to obtain a mixture; and
(iv) optionally adjusting the pH value of the mixture to a pH value in a range from 3.0 to 7.0.

10. The method according to claim 8, wherein the cereal beta-glucan is an oat beta-glucan.

11. The method according to claim 8, wherein the total beta-glucan content in the composition is from 0.1 to 10.0% by weight based on the total weight of the composition.

12. The method according to claim 8, wherein the inorganic salt consists of a monovalent or divalent metal and an anion of an inorganic acid or is a mixture of two or more of said inorganic salts; and/or the organic salt consists of a monovalent or divalent metal and an anion of an organic acid or is a mixture of two or more of said organic salts.

13. The method according to claim 8, wherein the added amount of salt in the composition is from 0.1 to 10% by weight based on the total weight of the composition.

14. (canceled)

15. A method for preparing food, food supplements, cosmetic, pharmaceutical or veterinary preparations comprising formulating the food, food supplements, cosmetic, pharmaceutical or veterinary preparations with the composition of claim 1.

16. The method according to claim 15, wherein the cosmetic, is for use in skin care, scalp care, hair care, nail care or for the prevention and/or the treatment of skin conditions, intolerant and sensitive skin, skin irritation, skin reddening, wheals, pruritus (itching), skin aging, wrinkle formation, loss of skin volume, loss of skin elasticity, pigment spots, pigment abnormalities, or dry skin.

17. The method according to claim 15, wherein the pharmaceutical or veterinary preparation is used in the prevention or treatment of dermatological or keratological diseases, of cardiovascular diseases, of allergic reactions, or of coronary heart disease, or for decreasing the level of LDL cholesterol and lipids in blood serum, for reducing blood pressure, for improving sensitivity to insulin, or for enabling the control of blood glucose levels.

18. The method according to claim 17, wherein the dermatological or keratological diseases are dermatological or keratological diseases having a barrier related inflammatory, immunoallergic, atherogenic, xerotic or hyperproliferative component or are associated with increased ROS production, wherein the dermatological or keratological disorders are selected from the group consisting of eczema, psoriasis, seborrhoea, dermatitis, erythema, pruritus (itching), otitis, inflammation, irritation, fibrosis, lichen planus, pityriasis rosea, pityriasis versicolor, autoimmune bullous diseases, urticarial, angiodermal and allergic skin reactions, and wound healing, and wherein the skin diseases associated with increased ROS production are selected from the group consisting of atopic dermatitis, neurodermitis, psoriasis, rosacea, acneiform eruptions, sebostasis and xerosis.

19. A food, food supplement, or cosmetic, pharmaceutical or veterinary preparation comprising the composition according to claim 1.

20. The composition according to claim 1 further comprising at least one cosmetic or pharmaceutical active substance selected from the group consisting of anti-inflammatories, antibacterial or antimycotic substances, substances having a reddening-alleviating or itch-alleviating action, lenitive substances, anti-dandruff, moisturizers and/or cooling agents, osmolytes, keratolytic substances, nurturing substances, anti-inflammatory, antibacterial or antimycotic substances, substances having a reddening-alleviating or itch-alleviating action, lenitive substances, antidandruff substances, or other active compounds selected from the group consisting of solvents, fragrances, antioxidants, preservatives, (metal) chelating agents, penetration enhancers, cosmetically and pharmaceutically additives or auxiliaries and mixtures thereof.

21. A method for retarding gelation of a cereal beta-glucan comprising composition comprising formulating the composition with at least one inorganic or organic salt, wherein the inorganic salt consists of a monovalent or divalent metal and an anion of an inorganic acid, or is a mixture of two or more of said inorganic salts; and/or the organic salt consists of a monovalent or divalent metal and an anion of an organic acid, or is a mixture of two or more of said organic salts.
